(19) United States
(12) Patent Application Publication
(10) Pub. No.: US 2025/0387458 A1
(43) Pub. Date: Dec. 25, 2025

(54) COACTIVE COMBINATIONS OF ANTIMICROBIALS WITH DISPERSINB

(71) Applicant: KANE BIOTECH INC., Winnipeg (CA)

(72) Inventors: Jeyachchandran VISVALINGAM, Winnipeg (CA); Nanda YAKANDAWALA, Winnipeg (CA); Suresh REGMI, Winnipeg (CA); Gordon GUAY, Winnipeg (CA)

(73) Assignee: KANE BIOTECH INC., Winnipeg (CA)

(21) Appl. No.: 19/316,587

(22) Filed: Sep. 2, 2025

Related U.S. Application Data
(63) Continuation of application No. 17/908,832, filed on Sep. 1, 2022.
(60) Provisional application No. 62/984,065, filed on Mar. 2, 2020.

(51) Int. Cl. A61K 38/47; A61K 31/155; A61K 31/785; A61P 31/04

(52) U.S. Cl. CPC A61K 38/47; A61K 31/155; A61K 31/785; A61P 31/04

(57) ABSTRACT

Particular antimicrobials are identified for efficacy in a composition with DispersinB to enhance biofilm dispersal and inactivation of biofilm embedded bacteria.

This application is a continuation of U.S. application Ser. No. 17/908,832, filed Sep. 1, 2022, which is the 371 Application of PCT/CA2021/050269, filed Mar. 2, 2021, which claims the benefit of U.S. Provisional Patent Application No. 62/984,065, filed Mar. 2, 2020, which is incorporated by reference herein, the entireties of all of which are hereby incorporated by reference in their entireties.

### FIELD OF INVENTION

The present invention relates to coactive DispersinB (DspB) formulations with antimicrobials and use of those formulations.

### BACKGROUND

DspB is an enzyme that is naturally produced by a periodontal disease-associated oral bacterium, Aggregatibacter actinomycetemcomitans. It specifically hydrolyses the glycosidic linkages of poly-beta 1, 6 N-acetylglucosamine (PNAG) leading to destabilization of biofilm structure and exposing biofilm-embedded bacteria. Purified recombinant DspB is shown to be active against diverse mammalian pathogens. In particular, PNAG is produced by a wide range of bacteria and fungi and is a key component in biofilm formation.

DspB cleaves PNAG, inhibiting bacterial adhesion and disperses the biofilm. This is especially useful for treating wounds and otic infections, which can become chronic due to the persistent nature of the bacterial biofilms. Once the biofilm is dispersed, the bacteria therein can be eradicated, and the infection can be remedied.

Bacteria is often eradicated or suppressed with antimicrobials or antibiotics.

However, the majority of broad-spectrum antimicrobials or disinfectants are surface active molecules that bind to and denature or inactivate proteins. For example:
Quaternary ammonium compounds (BKC and CPC) can irreversibly bind to protein and inactivate them (Maris. 1995. Rev. sci. tech. Off. int. Epiz., 14 (1), 47-55)
Chlorine compounds (Hypochlorite and chloramine) are generally electronegative, and therefore oxidises peptide links and denatures proteins. (Maris. 1995. Rev. sci. tech. Off. int. Epiz., 14 (1), 47-55)
Peracetic acid and hydrogen peroxide can oxidise and denature proteins and lipids of microorganisms, leading to disorganisation of the membrane. (Maris. 1995. Rev. sci. tech. Off. int. Epiz., 14 (1), 47-55)
Iodine compounds also interact with proteins and denature them. (Maris. 1995. Rev. sci. tech. Off. int. Epiz., 14 (1), 47-55)
Phenolic compounds also interact with proteins and denature them (Maris. 1995. Rev. sci. tech. Off. int. Epiz., 14 (1), 47-55)

As a result, when these antimicrobials are formulated with DspB, the DspB enzyme is typically inactivated before it can disperse the biofilm and release the bacteria. DspB is a protein/enzyme molecule that is susceptible to being inactivated by antimicrobial agents.

Previously, antimicrobial agents tested with DspB haven't been shown to work synergistically with DspB in a combination treatment or sequential treatment. In fact, the antimicrobial can be expected to inactivate the DspB.

It is a major challenge, therefore, to develop a formulation that enables DspB to function for a sufficient period of time to degrade PNAG based biofilms before a co-administered antimicrobial inactivates the DspB.

### SUMMARY OF INVENTION

In embodiments, the present invention provides uses of an antimicrobial in a composition with DspB to enhance biofilm dispersal and inactivation of biofilm embedded bacteria, the antimicrobial comprising one or more of polyhexamethylene biguanide, polyaminopropyl biguanide, alexidine dihydrochloride, carvacrol, thymol, cinnamaldehyde, chloroxylenol, octenidine dihydrochloride, benzalkonium chloride, cetylpyridinium chloride, providone iodine, neomycin sulphate, gentamicin sulfate, polymyxin B sulfate, metronidazole, mupirocin, gramicidin, rifampin, rifabutin, rifapentine, or vancomycin.

In other embodiments, the present invention provides compositions comprising: DspB and an antimicrobial for enhancing biofilm dispersal and inactivation of biofilm embedded bacteria, the antimicrobial comprising polyhexamethylene biguanide, polyaminopropyl biguanide, alexidine dihydrochloride, carvacrol, thymol, cinnamaldehyde, chloroxylenol, octenidine dihydrochloride, benzalkonium chloride, cetylpyridinium chloride, providone iodine, neomycin sulphate, gentamicin sulfate, polymyxin B sulfate, metronidazole, mupirocin, gramicidin, rifampin, rifabutin, rifapentine, Manuka honey or vancomycin.

The invention contemplates the usefulness of families of antimicrobials and antibiotics with DspB, wherein the utility of some members of the class has been demonstrated. Such classes of antimicrobials and antibiotics are set out in Table 4, below.

The present invention encompasses any and all combinations of any of the polyols, polymers, salts, preservatives, antimicrobials, and buffers described herein, for stabilization of DspB.

### BRIEF DESCRIPTION OF THE FIGURES

Exemplary embodiments are illustrated in referenced figures of the drawings. It is intended that the embodiments and figures disclosed herein are to be considered illustrative rather than restrictive.

FIG. 1 shows a bar graph illustrating the susceptibility of biofilm-embedded Staphylococcus epidermidis treated with or without polyhexamethylene biguanide (PHMB) and/or DspB at different concentrations.

FIG. 2 shows a bar graph illustrating the susceptibility of biofilm-embedded Escherichia coli treated with or without polyhexamethylene biguanide (PHMB) and/or DspB at different concentrations.

FIG. 3 shows a bar graph illustrating biofilm dispersal activity of DspB with or without polyhexamethylene biguanide (PHMB).

FIG. 4 shows a bar graph illustrating enzymatic activity of DspB with polyhexamethylene biguanide (PHMB) over time.

FIG. 5 shows a bar graph illustrating the susceptibility of biofilm-embedded S. epidermidis treated with or without polyaminopropyl biguanide (PAPB) and/or DspB at different concentrations.

FIG. 6 shows a bar graph illustrating the susceptibility of biofilm-embedded S. epidermidis treated with or without alexidine dihydrochloride (alexidine) and/or DspB at different concentrations.

FIG. 7 shows a bar graph illustrating the susceptibility of biofilm-embedded S. epidermidis treated with or without carvacrol and/or DspB at different concentrations.

FIG. 8 shows a bar graph illustrating the susceptibility of biofilm-embedded E. coli treated with or without carvacrol and/or DspB at different concentrations.

FIG. 9 shows a bar graph illustrating biofilm dispersal activity of DspB with or without carvacrol.

FIG. 10 shows a bar graph illustrating enzymatic activity of DspB with carvacrol over time.

FIG. 11 shows a bar graph illustrating the susceptibility of biofilm-embedded S. epidermidis treated with or without thymol and/or DspB at different concentrations.

FIG. 12 shows a bar graph illustrating the susceptibility of biofilm-embedded S. epidermidis treated with or without chloroxylenol and/or DspB at different concentrations.

FIG. 13 shows a bar graph illustrating the susceptibility of biofilm-embedded E. coli treated with or without chloroxylenol and/or DspB at different concentrations.

FIG. 14 shows a bar graph illustrating biofilm dispersal activity of DspB with or without chloroxylenol.

FIG. 15 shows a bar graph illustrating enzymatic activity of DspB with chloroxylenol over time.

FIG. 16 shows a bar graph illustrating the susceptibility of biofilm-embedded S. epidermidis treated with or without cinnamaldehyde and/or DspB at different concentrations.

FIG. 17 shows a bar graph illustrating the susceptibility of biofilm-embedded E. coli treated with or without cinnamaldehyde and/or DspB at different concentrations.

FIG. 18 shows a bar graph illustrating biofilm dispersal activity of DspB with or without cinnamaldehyde.

FIG. 19 shows a bar graph illustrating enzymatic activity of DspB with cinnamaldehyde over time.

FIG. 20 shows a bar graph illustrating the susceptibility of biofilm-embedded S. epidermidis treated with or without octenidine dihydrochloride and/or DspB at different concentrations.

FIG. 21 shows a bar graph illustrating the susceptibility of biofilm-embedded E. coli treated with or without octenidine dihydrochloride and/or DspB at different concentrations.

FIG. 22 shows a bar graph illustrating biofilm dispersal activity of DspB with or without octenidine dihydrochloride.

FIG. 23 shows a bar graph illustrating enzymatic activity of DspB with octenidine dihydrochloride over time.

FIG. 24 shows a bar graph illustrating biofilm dispersal activity of DspB with or without cetylpyridinium chloride.

FIG. 25 shows a bar graph illustrating biofilm dispersal activity of DspB with or without providone iodine.

FIG. 26 shows a bar graph illustrating biofilm dispersal activity of DspB with or without Gentamicin sulfate.

FIG. 27 shows a bar graph illustrating enzymatic activity of DspB with Gentamicin sulfate over time.

FIG. 28 shows a bar graph illustrating biofilm dispersal activity of DspB with or without Neomycin sulphate.

FIG. 29 shows a bar graph illustrating enzymatic activity of DspB with Neomycin sulphate over time.

FIG. 30 shows a bar graph illustrating biofilm dispersal activity of DspB with or without Polymyxin B sulfate.

FIG. 31 shows a bar graph illustrating enzymatic activity of DspB with Polymyxin B sulfate over time.

FIG. 32 shows a bar graph illustrating biofilm dispersal activity of DspB with or without Mupirocin.

FIG. 33 shows a bar graph illustrating enzymatic activity of DspB with Mupirocin over time.

FIG. 34 shows a bar graph illustrating biofilm dispersal activity of DspB with or without gramicidin.

FIG. 35 shows a bar graph illustrating enzymatic activity of DspB with gramicidin over time.

FIG. 36 shows a bar graph illustrating biofilm dispersal activity of DspB with or without Metronidazole.

FIG. 37 shows a bar graph illustrating enzymatic activity of DspB with Metronidazole over time.

FIGS. 38 to 69 show bar graphs illustrating biofilm inactivation, dispersal or enzymatic activity of DspB with antimicrobials in a gel formulation and biofilm dispersal activity over time.

FIG. 70 is a diagram showing packaging of DspB and antimicrobial containing gel in two compartments.

FIG. 71 shows a bar graph illustrating the susceptibility of biofilm-embedded S. epidermidis treated with or without Berberine hydrochloride and/or DspB at different concentrations.

FIG. 72 shows a bar graph illustrating the susceptibility of biofilm-embedded S. epidermidis treated with or without cinnamic acid and/or DspB at different concentrations.

FIG. 73 shows a bar graph illustrating enzymatic activity of DspB with cetylpyridinium chloride (CPC) over time.

FIG. 74 shows a bar graph illustrating biofilm dispersal activity of DspB with or without benzalkonium chloride (BZC).

FIG. 75 is a bar graph showing biofilm dispersal activity of DspB alone and with rifampin, rifapentine, and vancomycin.

FIG. 76 is a bar graph showing activity of DspB in the presence of rifampin.

FIG. 77 is a bar graph showing activity of DspB in the presence of rifapentine.

FIG. 78 is a bar graph showing activity of DspB in the presence of vancomycin.

FIG. 79 is a bar graph showing activity of DspB in the presence of manuka honey.

### DETAILED DESCRIPTION

The inventors have found that particular broad spectrum antimicrobials can be formulated with DspB without its inactivation for a sufficient period of time to enhance biofilm dispersal as well as inactivation of biofilm embedded bacteria. These formulations may be used for enhancing the activity of antimicrobials against biofilm embedded bacteria in chronic infections such as wounds, hard surfaces or in food processing or any other area where biofilm embedded bacteria contributing to human or animal infections.

Experiments were conducted against Gram− (E. coli) and Gram+ (S. epidermidis) bacteria to determine whether DspB can maintain its enzymatic activity when mixed with a broad spectrum antimicrobial. In addition to determining if the antimicrobials and enzyme were active when mixed together, tests were also conducted to determine whether how long DspB maintained its activity. Tests were conducted both in a liquid buffer system and a gel formulation to assess the activity of antimicrobial and enzyme combinations.

### Method 1: Method to Test Inactivation of Biofilm Embedded Bacteria Using Liquid Buffer Formulation

S. epidermidis or E. coli biofilm were each grown in 96-well plate and washed three times to remove planktonic cells. Then biofilm was treated using 50 mM phosphate or citrate buffer, (no treatment control), buffer solutions containing DspB at 10-200 μg/ml, buffer solution containing antimicrobial alone, and buffer solution DspB plus antimicrobial for varying exposure times (10 min-3 h). After treatments, biofilm embedded bacteria was dispersed, diluted serially and plated on Tryptic soy agar plates. Data was analyzed using ANOVA and Tukey's test. Mean values were considered significantly different when P value was ≤0.05.

### Method 2: Method to Test Inactivation of Biofilm Embedded Bacteria Using Gel Formulation

S. epidermidis biofilm was grown in 96-well plate and washed three times to remove planktonic cells. Then biofilm was treated using different DspB and antimicrobial gel formulations for appropriate treatment duration (10 min-24 h). After treatment, gel was removed and washed 3 times using sterile water to remove excess gel. Then biofilm embedded bacteria was dispersed, diluted serially and plated on Tryptic soy agar plates.

### Method 3: Method to Test Compatibility Between Antimicrobials or Antibiotics and DspB Using Biofilm Dispersal Assay

S. epidermidis biofilm was grown in 96-well plate and washed three times using sterile water to remove planktonic cells. Antimicrobials or antibiotics and DspB formulations were prepared in 50 mM phosphate or citrate buffer and stored at 37° C. for 2 h and used for treating biofilm at 0 and 2 h. Biofilm was treated using antimicrobials or antibiotics and DspB formulations for 10 min. After treatments, solutions were removed and washed 3 times to remove dispersed biofilm. Remaining biofilms in 96-well plates were stained using Crystal violet, unbound crystal violet was removed by washing the wells with sterile water, the biofilm bound crystal violet was eluted using 33% acetic acid, and the absorbance at 620 nm was taken using a microwell plate reader. Percent biofilm removal was calculated. Data was analyzed using ANOVA and Tukey's test. Mean values were considered significantly different when P value was ≤0.05.

### Method 4: Method to Test Compatibility Between Antimicrobials or Antibiotics and DspB Using Enzymatic Assay

DspB (200 μg/mL) and antimicrobial agent/antibiotic stocks were prepared in respective buffers. 10 mM paranitrophenol (PNP) in 400 mM sodium carbonate, and the buffer (same DspB stock) was used as the standard solution, and negative control, respectively. DspB stock and antimicrobial stock were mixed in equal proportions, and then 4×5 μl samples were taken at defined time points (0, 5, 10, 20, 30, 60, and 120 min) and used to measure the DspB enzyme activity. DspB enzymatic activity was measured using β-N-Acetylglucosaminidase assay kit from Sigma (product code CS0780) in 96-well microtiter plate following the manufacturer's instructions. The plate was incubated for 30 minutes before terminating the reaction. Data analysis was done in Excel. Student T test (paired) was performed for each time point separately using DspB activity in mixture and in control sample as two set of variables.

### Method 5: Method to Test Biofilm Dispersal by DspB Gel Formulations

S. epidermidis biofilm was grown in 96-well plate and washed three times to remove planktonic cells. Then biofilm was treated using different DspB formulations appropriate treatment duration (10 min-1 h). After treatments, gels were removed and washed 3 times with sterile distilled water to remove excess gel and dispersed biofilm. Remaining biofilms in 96-well plates were stained using Crystal violet and unbound crystal violet was removed by washing the wells with sterile water, the biofilm bound crystal violet was eluted using 33% acetic acid, and the absorbance at 620 nm was taken using a microwell plate reader.

### Method 6: Method to Test Compatibility Between Antimicrobials or Antibiotics and DspB in Gel Using Enzymatic Assay

DspB gel containing 200 μg/ml DspB, 16% Poloxamer 405 (PF127), 50 mM Phosphate buffer or citrate, 100 mM NaCl, pH 5.9, and 2× concentrated antimicrobial agent/antibiotics in 16% Poloxamer 405, 50 mM Phosphate buffer, 100 mM NaCl, pH 5.9 were mixed in equal proportions. 4×5 μL aliquots were withdrawn at 0, 5, 10, 20, 30, 60, and 120 min for measurement of DspB enzymatic activity. DspB enzymatic activity was measured using β-N-acetylglucosaminidase assay kit from Sigma (product code CS0780) in 96-well microtiter plate following the manufacturer's instructions. The plate was incubated for 30 minutes before terminating the reaction. Data analysis was done in Excel. Student T test (paired) was performed for each time point separately using DspB activity in mixture and in control sample as two set of variables.

The antimicrobial classes tested include: biguanides, monoterpenes/plant extracts or essential oil components, halophenols, phenylpropanoid, cationic surfactants, quaternary ammonium compounds, iodine compounds. Examples of specific biguanides tested include polyhexamethylene biguanide (PHMB), polyaminopropyl biguanide (PAPB), and alexidine. Examples of specific monoterpenes/plant extracts or essential oil components tested include carvacrol and thymol. An example of a halophenols tested includes chloroxylenol. An example of a phenylpropanoid tested includes cinnamaldehyde. An example of cationic surfactants tested includes octenidine dihydrochloride. Examples for quaternary ammonium compounds are cetylpyridinium chloride and benzalkonium chloride. An example of iodine compound is providone iodine.

Antibiotics that are commonly used for wound care application, and tested herein, include mupirocin, neomycin sulphate, gentamicin sulfate, polymyxin B sulfate, gramicidin, metronidazole, rifampin, rifapentine and vancomycin.

Tests were conducted to evaluate a) enhancement of inactivation of biofilm embedded bacteria by antimicrobial agents in the presence of DspB in buffer or gel (16% Poloxamer 405, 50 mM Phosphate/citrate buffer, 100 mM NaCl, pH 5.9) formulation (Method 1 and Method 2, respectively), and b) stability of DspB in the presence of antimicrobial agents in buffer or gel formulation were evaluated using biofilm dispersal assay (Method 3 and Method 5) and using enzymatic assay (Method 4 and 6), respectively.

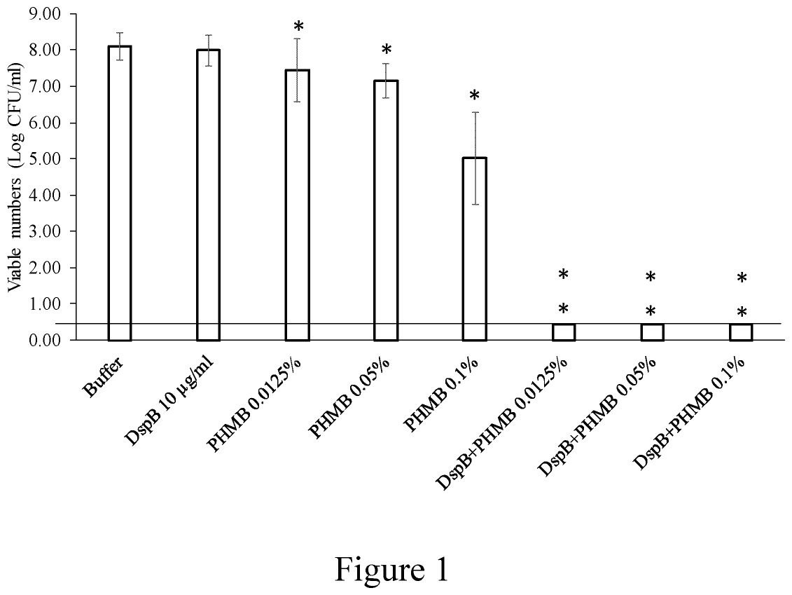

Initial testing done with DspB and PHMB in buffer system showed that 10 μg/ml (FIG. 1) and 20 μg/ml yielded similar inactivation of biofilm embedded bacteria and 10 min treatment was sufficient to disperse biofilms. When DspB was added to gel, colloidal matrix may delay diffusion and biofilm dispersal activity of DspB. Thus, for both buffer and gel formulations, DspB concentration was fixed at 20 μg/ml for most cases.

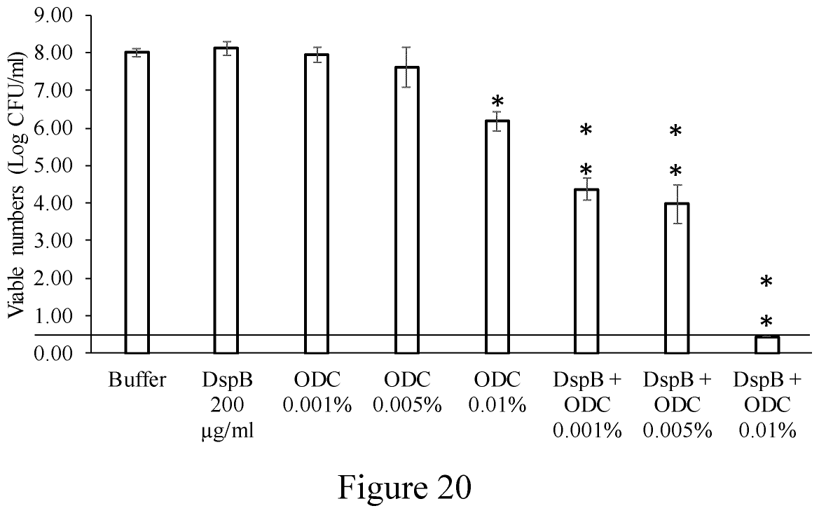

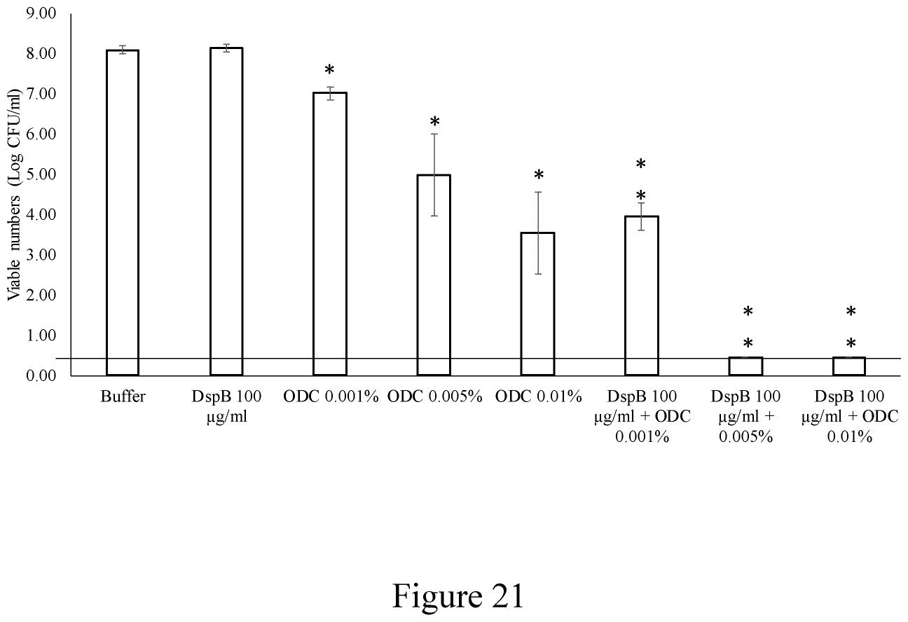

Some antimicrobials like octenidine dihydrochloride imparted negative effect on DspB when tested at 20 μg/ml. When 20 μg/ml DspB was mixed with this antimicrobial and biofilms were treated for 10 min, no biofilm dispersal was observed (data not presented). Thus, concentration of DspB was increased to 5× or 10× for further testing. DspB at 100-200 enhanced activity of octenidine dihydrochloride (FIGS. 20 and 21).

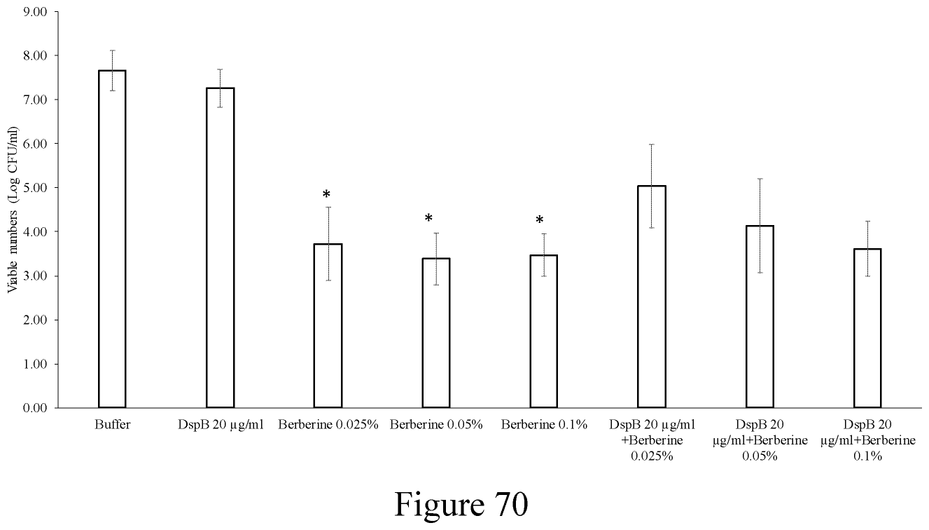

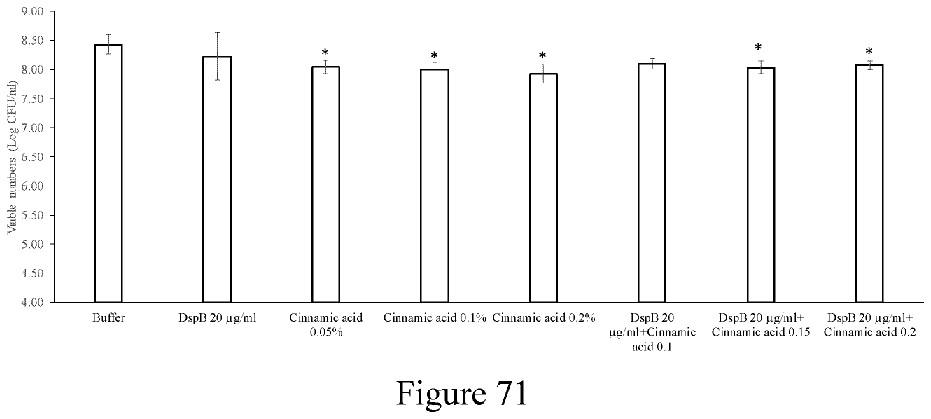

DspB at 20 μg/ml did not enhance activity of berberine hydrochloride (FIG. 70). Rather, inactivation of biofilm embedded bacteria by berberine hydrochloride and 20 μg/ml DspB treatment was lower than berberine hydrochloride treatment alone (antagonism). Cinnamic acid treatment with DspB at 20 μg/ml did not show any enhancement of bacterial inactivation in biofilm (FIG. 71). Thus, further testing was not conducted with these compounds.

A summary of the antimicrobials or antibiotics tested with DspB in buffer formulations is set out in Table 1, and Examples 1 to 16. A summary of the antimicrobials or antibiotics tested with DspB in gel formulations is set out in Table 2 and Examples 17 to 35.

Based on the experimental results described herein, it is shown that at least polyhexamethylene biguanide, polyaminopropyl biguanide, alexidine dihydrochloride, carvacrol, thymol, cinnamaldehyde, chloroxylenol, octenidine dihydrochloride, benzalkonium chloride, cetylpyridinium chloride, providone iodine, neomycin sulphate, gentamicin sulfate, polymyxin B sulfate, gramicidin, metronidazole mupirocin, refampin, refapentine, vancomycin, Manuka honey and gramicidin are useful in allowing DspB to maintain its enzymatic activity for a reasonable amount of time when mixed. Thus, it was found that these antimicrobials and antibiotics in combination with DspB are useful in enhancing biofilm dispersal and enhancing inactivation of bacteria embedded therein.

### Example 1—Use of Polyhexamethylene Biguanide (PHMB)

Polyhexamethylene biguanide (PHMB), also referred to as polyhexanide, is a polymer typically used as a disinfectant and antiseptic. The chemical structure of PHMB is:

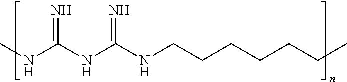

The present invention provides uses of PHMB with DspB, and compositions thereof, to enhance biofilm dispersal and inactivation of biofilm embedded bacteria. The PHMB with DspB may be applied in a liquid buffer formulation, a gel formulation, or in a manner known in the art.

In an embodiment, the concentration of PHMB is up to 0.15%. In a preferred embodiment, the concentration of PHMB is between 0.00313 and 0.1%. In a further preferred embodiment, when the composition is a gel formulation, the concentration of PHMB is about 0.1%.

In another embodiment, the concentration of DspB is between 10 and 20 μg/ml. In a preferred embodiment, the concentration of DspB is about 20 μg/ml.

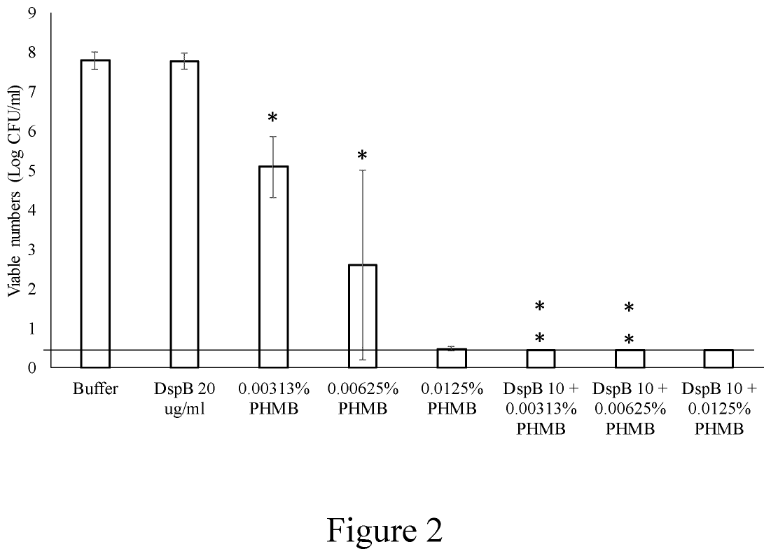

Inactivation of biofilm embedded S. epidermidis and E. coli were tested using Method 1. When PHMB was added with DspB, it enhanced (P<0.05) activity of PHMB against both S. epidermidis (see FIG. 1) and E. coli (see FIG. 2).

FIG. 1 is bar graph showing the increased susceptibility of biofilm-embedded S. epidermidis treated with no antimicrobials in 50 mM Phosphate buffer (buffer), DspB at 10 μg/ml, PHMB at 0.0125, 0.05, and 0.1%, DspB+PHMB at 0.0125%, DspB+PHMB 0.05% and DspB and PHMB at 0.1% for 10 min at 37° C. Horizontal line was the detection limit of viable numbers. *Indicate significant reduction (P≤0.05) in viable numbers compared to buffer or DspB control. **indicate significant (P≤0.05) reduction in viable count compared to corresponding PHMB or DspB treatment alone. Note that DspB does not kill bacteria but instead dissolves the PNAG based biofilm that they reside in.

FIG. 2 is bar graph showing the increased susceptibility of biofilm-embedded E. coli treated with no antimicrobials in 50 mM phosphate buffer (buffer), DspB at 20 μg/ml PHMB at 0.0125, 0.05, and 0.1%, +PHMB at 0.0125%, DspB+PHMB 0.05% and DspB and PHMB at 0.1% for 10 min at 37° C. Horizontal line was the detection limit of viable numbers. *Indicate significant reduction (P≤0.05) in viable numbers compared to buffer or DspB control. **indicate significant (P≤0.05) reduction in viable count compared to corresponding PHMB or treatment alone.

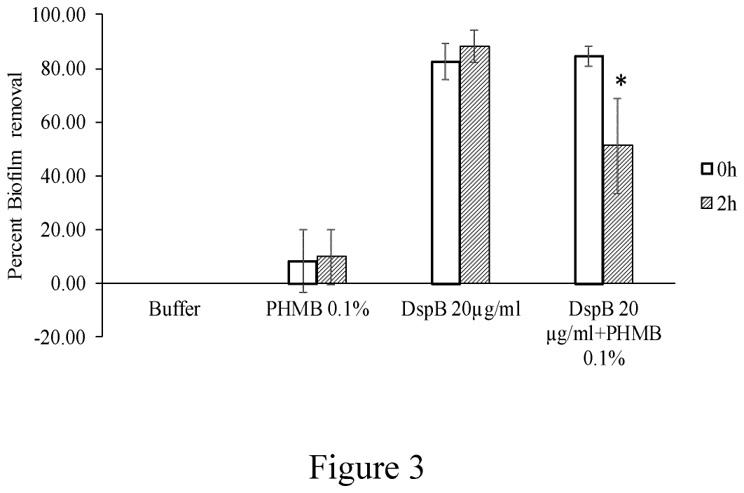

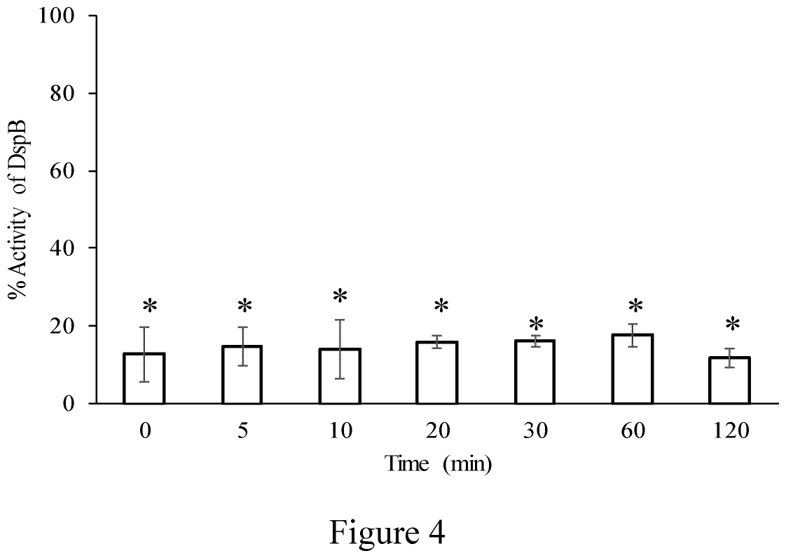

Compatibility between DspB and PHMB was tested using biofilm dispersal assay (Method 3) and enzymatic assay (Method 4). Results showed that ≥50% biofilm dispersal was observed when DspB and PHMB were added together (see FIG. 3). This result and the enzymatic assay result (see FIG. 4) suggest that DspB retained its activity for up to 2 h in the presence of PHMB.

FIG. 3 is a bar graph showing biofilm dispersal activity of 50 mM phosphate buffer (buffer), PHMB 0.1% alone, 20 μg/ml DspB alone and 20 μg/ml DspB plus 20 μg/ml polyhexamethylene biguanide (PHMB). Results showed that DspB retained its activity for up to 2 h in the presence of PHMB. *Indicate significant reduction (P≤0.05) in biofilm dispersal compared to DspB control.

FIG. 4 is a bar graph showing activity of DspB in the presence of 0.1% PHMB. DspB concentration used in mixture (DspB+PHMB) was 100 μg/mL in 50 mM phosphate buffer and it's % activity was calculated compared to the activity of 100 μg/mL DspB (considered 100% activity) as a control sample. Results showed that DspB was active for up to 2 h. *Indicate significant reduction (P≤0.05) in DspB activity compared to DspB control.

### Example 2—Use of Polyaminopropyl Biguanide (PAPB)

Polyaminopropyl Biguanide (PAPB) is commonly used as a disinfectant and a preservative. The chemical structure of PAPB is:

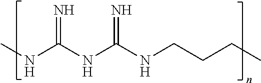

The present invention provides uses of PAPB with DspB, and compositions thereof, to enhance biofilm dispersal and inactivation of biofilm embedded bacteria. The PAPB with DspB may be applied in a liquid buffer formulation, a gel formulation, or in a manner known in the art.

In an embodiment, the concentration of PAPB is up to 0.25%. In a preferred embodiment, the concentration of PHMB is between 0.05 and 0.2%. In a further preferred embodiment, when the composition is a gel formulation, the concentration of PHMB is about 0.1%.

In another embodiment, the concentration of DspB is between 10 and 20 μg/ml. In a preferred embodiment, the concentration of DspB is about 20 μg/ml.

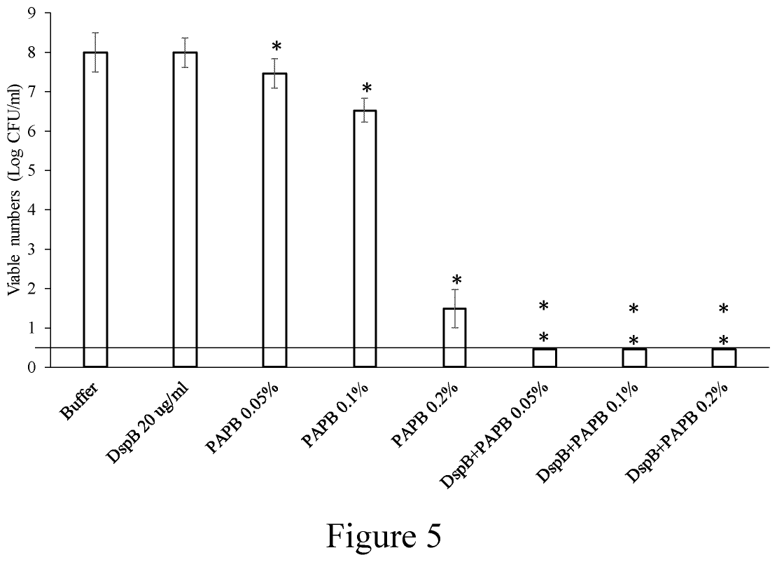

Inactivation of biofilm embedded S. epidermidis was tested using Method 1. When PAPB was added with DspB, DspB enhanced (P<0.05) activity of PAPB against S. epidermidis (see FIG. 5).

FIG. 5 is bar graph showing the increased susceptibility of biofilm-embedded S. epidermidis treated with no antimicrobials in 50 mM citrate buffer (buffer), DspB at 20 μg/ml, PAPB at 0.05, 0.1, and 0.2%, DspB+PAPB at 0.05%, DspB+PAPB 0.1% and DspB and PAPB at 0.2% for 10 in at 37° C. Horizontal line was the detection limit of viable numbers. *Indicate significant reduction (P≤0.05) in viable numbers compared to buffer or DspB control. **indicate significant (P≤0.05) reduction in viable count compared to corresponding PAPB or DspB treatment alone.

### Example 3—Use of Alexidine Dihydrochloride (Alexidine)

Alexidine dihydrochloride (alexidine) is a biguanide class antimicrobial with a chemical structure of:

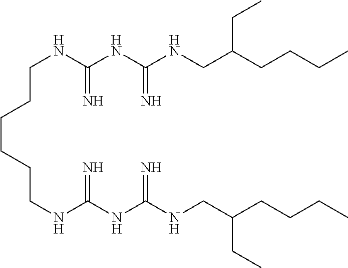 [SMILES: CCCCC(CC)CNC(=N)NC(=N)NCCCCCCNC(=N)NC(=N)NCC(CC)CCCC]

The present invention provides uses of alexidine with DspB, and compositions thereof, to enhance biofilm dispersal and inactivation of biofilm embedded bacteria. The alexidine with DspB may be applied in a liquid buffer formulation, a gel formulation or in a manner known in the art.

In an embodiment, the concentration of alexidine is up to 0.1%. In a preferred embodiment, the concentration of alexidine is between 0.0125 and 0.05%.

In another embodiment, the concentration of DspB is between 10 and 20 μg/ml. In a preferred embodiment, the concentration of DspB is about 20 μg/ml.

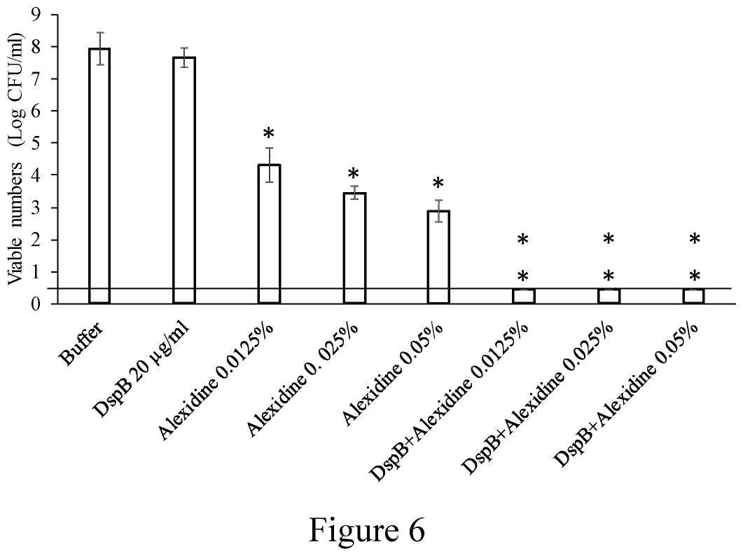

Inactivation of biofilm embedded S. epidermidis was tested using Method 1. When Alexidine was added with DspB, DspB enhanced (P<0.05) activity of alexidine against S. epidermidis (see FIG. 6).

FIG. 6 is bar graph showing the increased susceptibility of biofilm-embedded S. epidermidis treated with no antimicrobials in 50 mM phosphate buffer (buffer), DspB at 20 μg/ml, alexidine at 0.0125, 0.025, and 0.05%, DspB+alexidine at 0.0125%, DspB+alexidne 0.025% and DspB and alexidine at 0.05% for 10 min at 37° C. Horizontal line was the detection limit of viable numbers. *Indicate significant reduction (P≤0.05) in viable numbers compared to buffer or DspB control. **indicate significant (P≤0.05) reduction in viable count compared to corresponding alexidine or DspB treatment alone.

### Example 4—Use of Carvacrol

Carvacrol, also referred to as cymophenol, is a monoterpenoid phenol often found in essential oils. The chemical structure of carvacrol is:

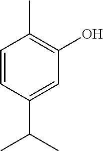

The present invention provides uses of carvacrol with DspB, and compositions thereof, to enhance biofilm dispersal and inactivation of biofilm embedded bacteria. The carvacrol with DspB may be applied in a liquid buffer formulation, a gel formulation or in a manner known in the art.

In an embodiment, the concentration of carvacrol is up to 0.15%. In a preferred embodiment, the concentration of carvacrol is between 0.05 and 0.1%. In a further preferred embodiment, when the composition is a gel formulation, the concentration of carvacrol is about 0.1%.

In another embodiment, the concentration of DspB is up to 100 μg/ml. In a preferred embodiment, the concentration of DspB is about 20 μg/ml.

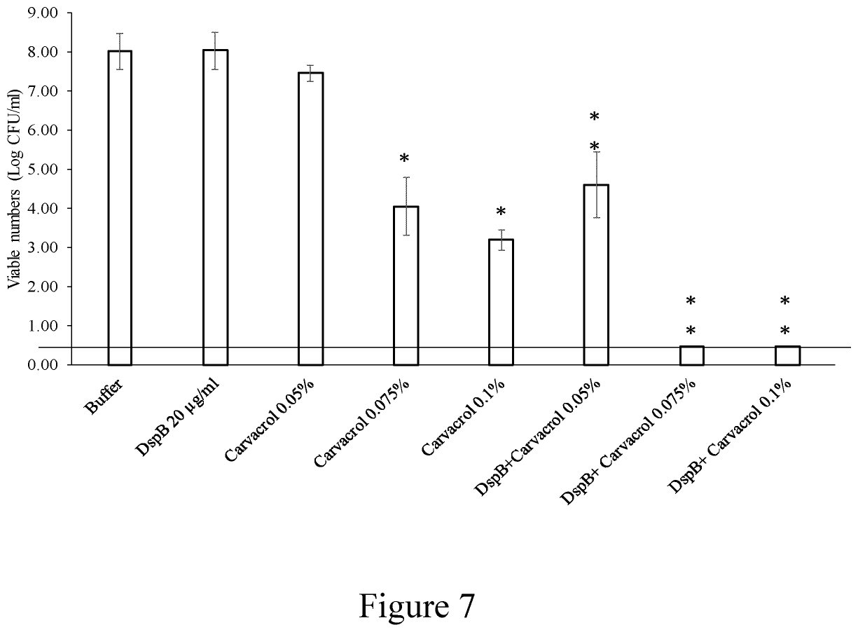

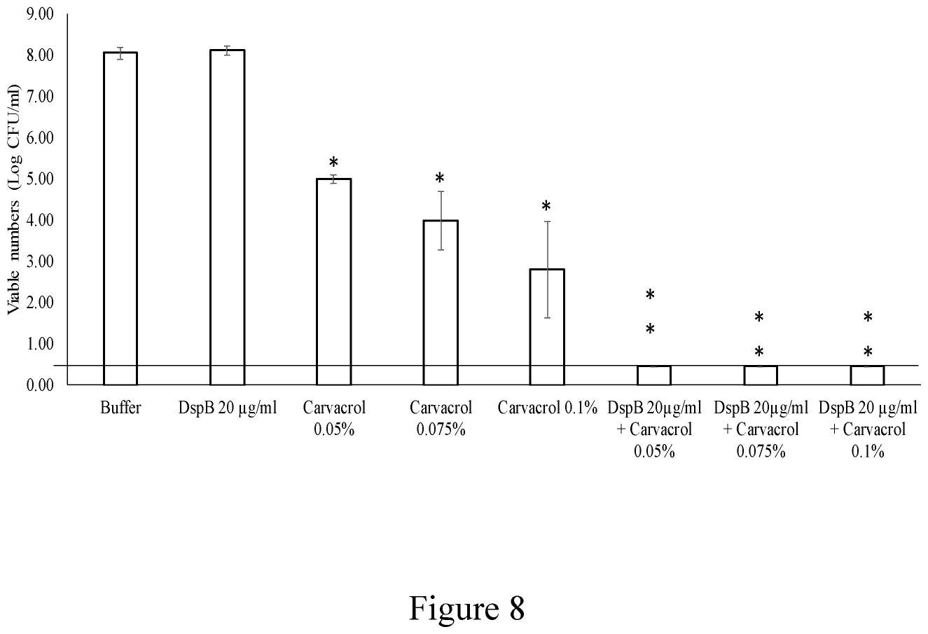

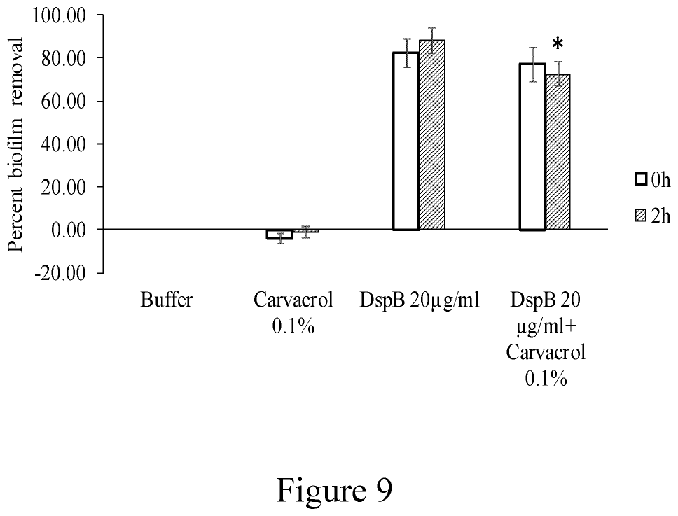

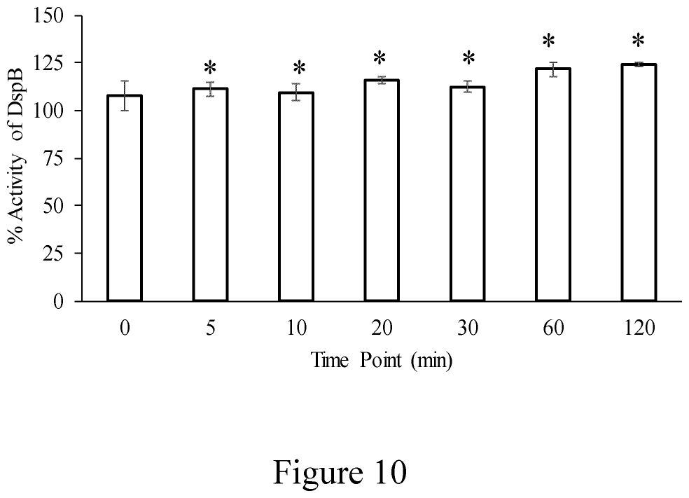

Inactivation of biofilm embedded S. epidermidis and E. coli were tested using Method 1. When carvacrol was added with DspB, DspB enhanced (P<0.05) activity of carvacrol against both S. epidermidis (see FIG. 7) and E. coli (see FIG. 8). Compatibility between DspB and carvacrol was tested using biofilm dispersal assay (Method 3) and enzymatic assay (Method 4). Results showed that ≥73% biofilm dispersal was observed when DspB and carvacrol were added together (see FIG. 9). This results and enzymatic assay (see FIG. 10) suggest that DspB retained its activity for up to 2 h in the presence of carvacrol.

FIG. 7 is bar graph showing the increased susceptibility of biofilm-embedded S. epidermidis treated with no antimicrobials in 50 mM citrate buffer (buffer), DspB at 20 μg/ml, carvacrol at 0.05, 0.075, and 0.1%, DspB+carvacrol at 0.05%, DspB+carvacrol 0.075% and DspB and carvacrol at 0.1% for 10 min at 37° C.. Horizontal line was the detection limit of viable numbers. *Indicate significant reduction (P≤0.05) in viable numbers compared to buffer or DspB control. **indicate significant (P≤0.05) reduction in viable count compared to corresponding carvacrol or DspB treatment alone.

FIG. 8 is bar graph showing the increased susceptibility of biofilm-embedded E. coli treated with no antimicrobials 50 mM citrate buffer (buffer), DspB at 20 μg/ml, carvacrol at 0.05, 0.075, and 0.1%, DspB+carvacrol at 0.05%, DspB+carvacrol 0.075% and DspB and carvacrol at 0.1% for 10 min at 37° C.. Horizontal line was the detection limit of viable numbers. *Indicate significant reduction (P≤0.05) in viable numbers compared to buffer or DspB control. **indicate significant (P≤0.05) reduction in viable count compared to corresponding carvacrol or DspB treatment alone.

FIG. 9 is a bar graph showing biofilm dispersal activity of 50 mM citrate buffer (buffer), 0.1% carvacrol alone, 20 μg/ml DspB alone and 20 μg/ml DspB plus 0.1% carvacrol. Results showed that DspB retained its activity for up to 2 h in the presence of carvacrol. *Indicate significant change (P≤0.05) in biofilm dispersal compared to DspB control.

FIG. 10 is a bar graph showing activity of DspB in the presence of 0.1% carvacrol. DspB concentration used in mixture (DspB+carvacrol) was 100 μg/mL in 50 mM citrate buffer and it's % activity was calculated compared to the activity of 100 μg/mL DspB (considered 100% activity) as a control sample. Results showed that DspB was active for up to 2 h. *Indicate significant increase (P≤0.05) in DspB activity compared to DspB control.

### Example 5—Use of Thymol

Thymol is a natural monoterpenoid phenol derivative of cymene with antiseptic properties. The chemical structure of thymol is:

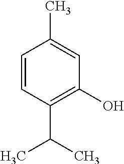

The present invention provides uses of thymol with DspB, and compositions thereof, to enhance biofilm dispersal and inactivation of biofilm embedded bacteria. The thymol with DspB may be applied in a liquid buffer formulation, a gel formulation, or in a manner known in the art.

In an embodiment, the concentration of thymol is up to 0.15%. In a preferred embodiment, the concentration of alexidine is between 0.05 and 0.1%.

In another embodiment, the concentration of DspB is between 10 and 20 μg/ml. In a preferred embodiment, the concentration of DspB is about 20 μg/ml.

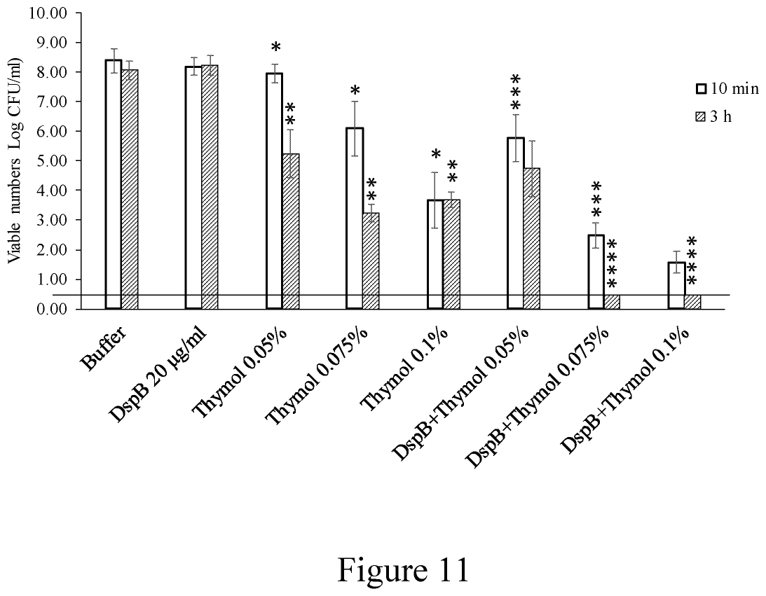

Inactivation of biofilm embedded S. epidermidis was tested using Method 1. When thymol was added with DspB enhanced (P<0.05) activity of thymol against S. epidermidis (see FIG. 11).

FIG. 11 is bar graph showing the increased susceptibility of biofilm-embedded S. epidermidis treated with no antimicrobials in 50 mM citrate buffer (buffer), DspB at 20 μg/ml, thymol at 0.05, 0.075, and 0.1%, DspB+thymol at 0.05%, DspB+thymol 0.075% and DspB and thymol at 0.1% for 10 min or 3 h at 37° C. Horizontal line was the detection limit of viable numbers. Indicate significant reduction (P<0.05) in viable numbers compared to buffer control for 10 min treatment. **Indicate significant reduction (P<0.05) in viable numbers compared to buffer control after 3 h treatment. ***indicate significant (P≤0.05) reduction in viable count compared to corresponding thymol treatment alone for 10 min. ****indicate significant (P≤0.05) reduction in viable count compared to corresponding thymol treatment alone for 3 h.

### Example 6—Use of Chloroxylenol

Chloroxylenol, also referred to as para-chloro-meta-xylenol, is an antiseptic and disinfectant. The chemical structure of chloroxylenol is:

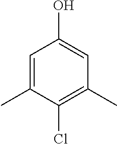

The present invention provides uses of chloroxylenol with DspB, and compositions thereof, to enhance biofilm dispersal and inactivation of biofilm embedded bacteria. The chloroxylenol with DspB may be applied in a liquid buffer formulation, a gel formulation, or in a manner known in the art.

In an embodiment, the concentration of thymol is up to 0.25%. In a preferred embodiment, the concentration of alexidine is between 0.05 and 0.2%.

In another embodiment, the concentration of DspB is up to 100 μg/ml. In a preferred embodiment, the concentration of DspB is about 20 μg/ml.

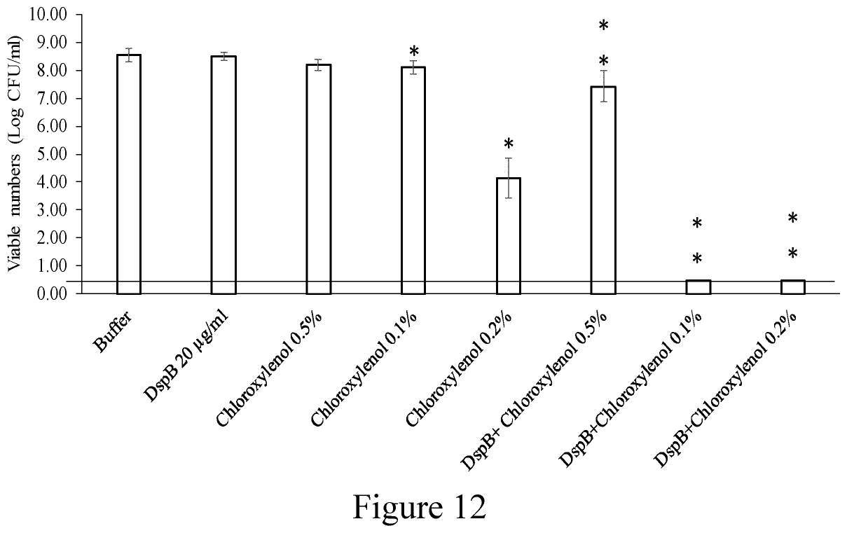

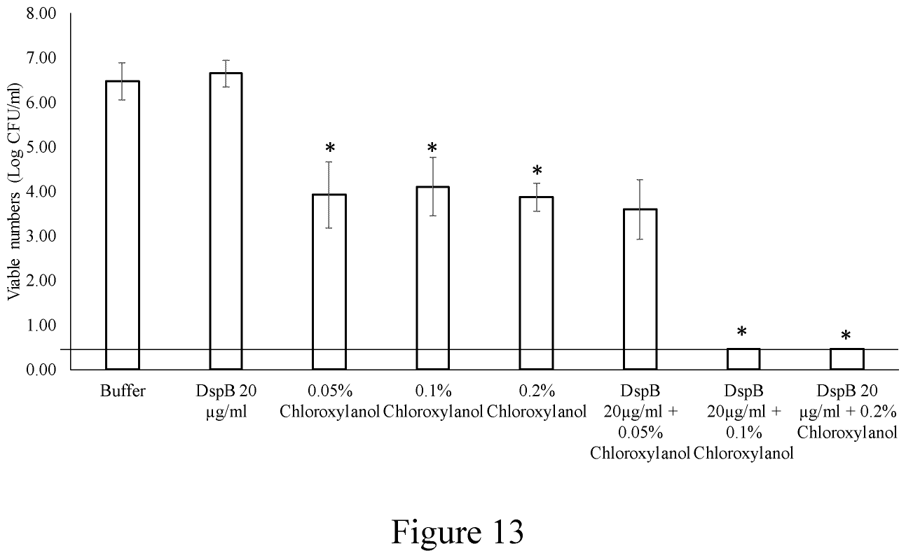

Inactivation of biofilm embedded S. epidermidis and E. coli were tested using Method 1. When chloroxylenol was added with DspB, DspB enhanced (P<0.05) activity of chloroxylenol against both S. epidermidis (see FIG. 12) and E. coli (see FIG. 13). Compatibility between DspB and chloroxylenol was tested using biofilm dispersal assay (Method 3) and enzymatic assay (Method 4). Results showed that ≥74% biofilm dispersal was observed when DspB and chloroxylanol were added together (FIG. 9). This results and enzymatic assay (see FIG. 10) suggest that DspB retained its activity for up to 2 h in the presence of chloroxylanol.

FIG. 12 is bar graph showing the increased susceptibility of biofilm-embedded S. epidermidis treated with no antimicrobials in 50 mM citrate buffer (buffer), DspB at 20 μg/ml, chloroxylenol at 0.05, 0.1, and 0.2%, DspB+chloroxylenol at 0.05%, DspB+chloroxylenol 0.1% and DspB and chloroxylenol at 0.2% for 10 min at 37° C. Horizontal line was the detection limit of viable numbers. *Indicate significant reduction (P≤0.05) in viable numbers compared to buffer or DspB control. **indicate significant (P≤0.05) reduction in viable count compared to corresponding chloroxylenol or DspB treatment alone.

FIG. 13 is bar graph showing the increased susceptibility of biofilm-embedded E. coli treated with no antimicrobials in 50 mM citrate buffer (buffer), DspB at 20 μg/ml, chloroxylenol at 0.05, 0.1, and 0.2%, DspB+chloroxylenol at 0.05%, DspB+chloroxylenol 0.1% and DspB and chloroxylenol at 0.2% for 10 min at 37° C. Horizontal line was the detection limit of viable numbers. *Indicate significant reduction (P≤0.05) in viable numbers compared to buffer or DspB control. **indicate significant (P≤0.05) reduction in viable count compared to corresponding chloroxylenol or DspB treatment alone.

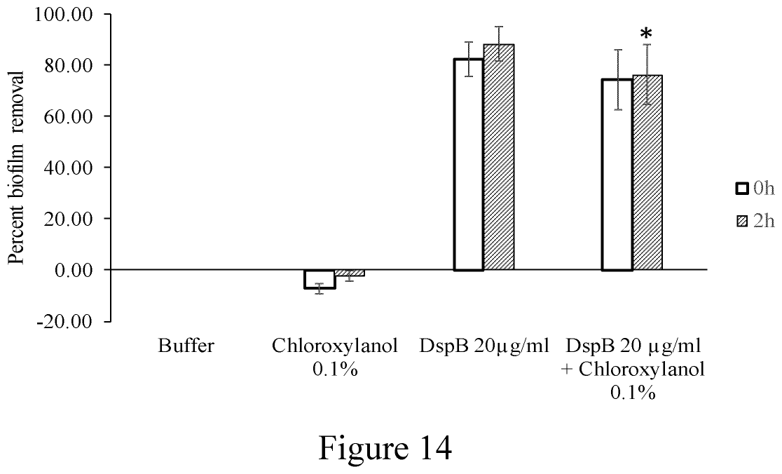

FIG. 14 is a bar graph showing biofilm dispersal activity of 50 mM citrate buffer (buffer), 0.1% chloroxylenol alone, 20 μg/ml DspB alone and 20 μg/ml DspB plus 0.1% chloroxylenol. Results showed that DspB retained its activity for up to 2 h in the presence of chloroxylenol. *Indicate significant reduction (P≤0.05) in biofilm dispersal compared to DspB control.

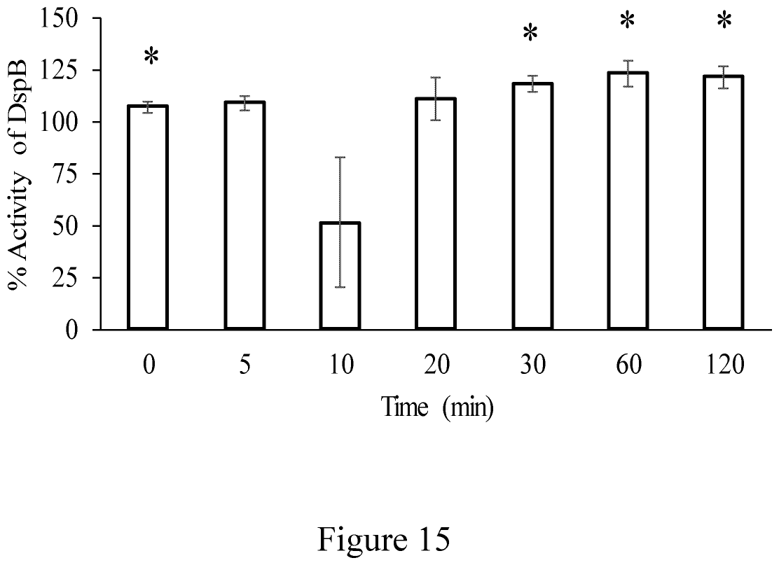

FIG. 15 is a bar graph showing activity of DspB in the presence of 0.1% chloroxylenol. DspB concentration used in mixture (DspB+chloroxylenol) was 100 μg/mL in 50 mM citrate buffer and it's % activity was calculated compared to the activity of 100 μg/mL DspB (considered 100% activity) as a control sample. Results showed that DspB was active for up to 2 h. *Indicate significant change (P≤0.05) in DspB activity compared to DspB control.

### Example 7—Use of Cinnamaldehyde

Cinnamaldehyde is an organic compound that gives cinnamon its flavour and colour. The chemical structure of cinnamaldehyde is:

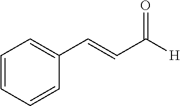

The present invention provides uses of cinnamaldehyde with DspB, and compositions thereof, to enhance biofilm dispersal and inactivation of biofilm embedded bacteria. The cinnamaldehyde with DspB may be applied in a liquid buffer formulation, a gel formulation, or in a manner known in the art.

In an embodiment, the concentration of cinnamaldehyde is up to 0.25%. In a preferred embodiment, the concentration of cinnamaldehyde is between 0.05 and 0.2%. In a further preferred embodiment, when the composition is a gel formulation, the concentration of cinnamaldehyde is about 0.2%.

In another embodiment, the concentration of DspB is up to 250 μg/ml. In a preferred embodiment, the concentration of DspB is between 100 and 200 μg/ml.

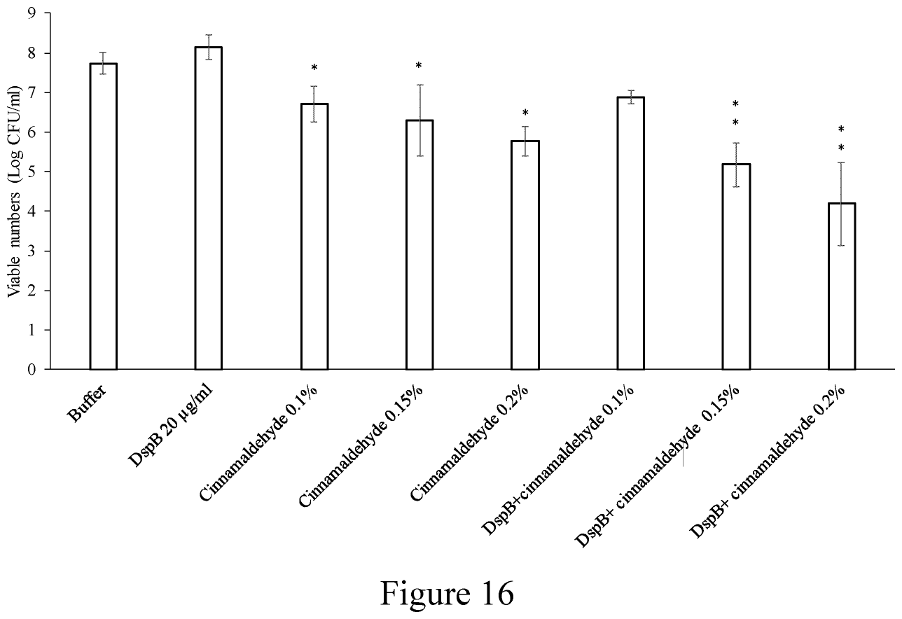

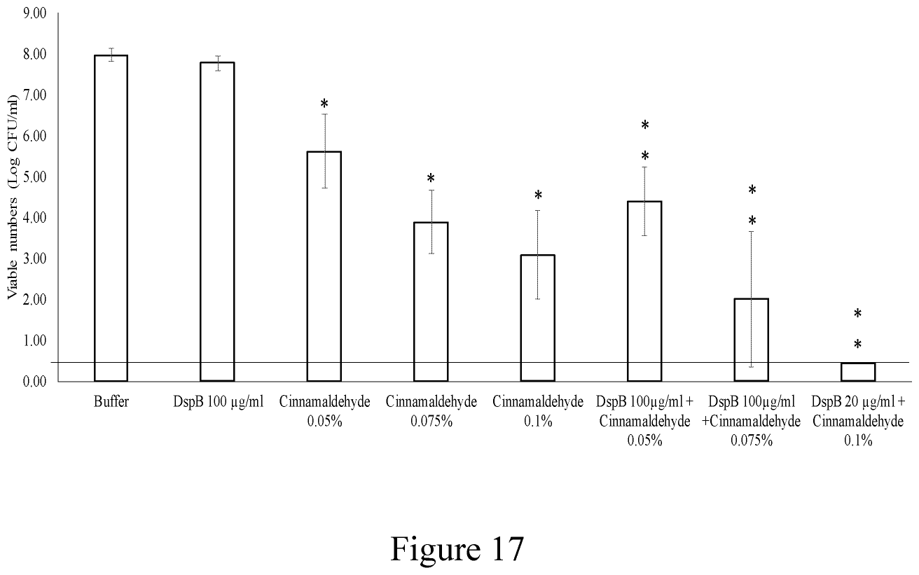

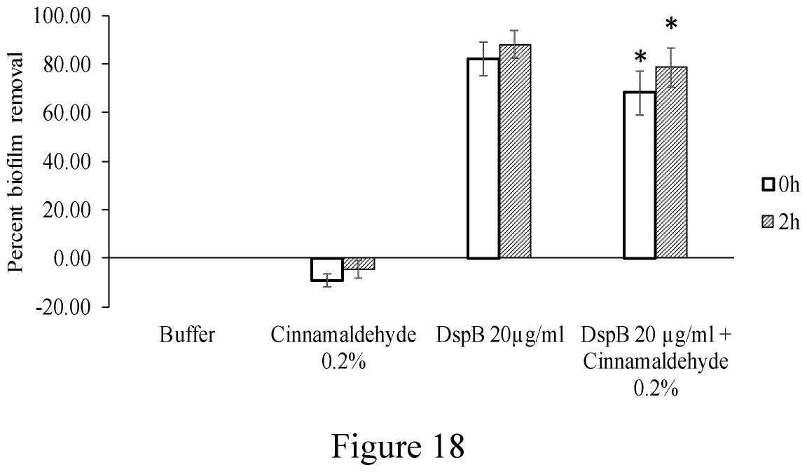

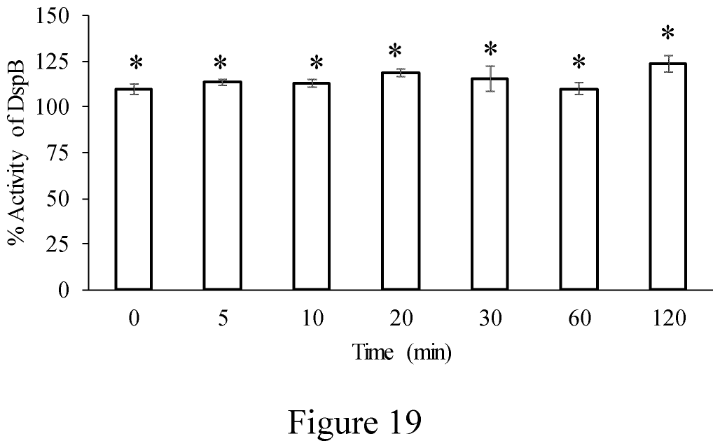

Inactivation of biofilm embedded S. epidermidis and E. coli were tested using Method 1. When cinnamaldehyde was added with DspB, DspB enhanced (P<0.05) activity of cinnamaldehyde against both S. epidermidis (see FIG. 16) and E. coli (see FIG. 17). Compatibility between DspB and cinnamaldehyde was tested using biofilm dispersal assay (Method 3) and enzymatic assay (Method 4). Results showed that ≥78% biofilm dispersal was observed when DspB and cinnamaldehyde were added together (see FIG. 18). This results and enzymatic assay (see FIG. 19) suggest that DspB retained its activity for up to 2 h in the presence of cinnamaldehyde.

FIG. 16 is bar graph showing the increased susceptibility of biofilm-embedded S. epidermidis treated with no antimicrobials in 50 mM citrate buffer (buffer), DspB at 20 μg/ml, cinnamaldehyde at 0.1, 0.15, and 0.2%, DspB+cinnamaldehyde at 0.1%, DspB+cinnamaldehyde 0.15% and DspB and cinnamaldehyde at 0.2% for 3 h at 37° C. Horizontal line was the detection limit of viable numbers. *Indicate significant reduction (P≤0.05) in viable numbers compared to buffer or DspB control. **indicate significant (P≤0.05) reduction in viable count compared to corresponding cinnamaldehyde or DspB treatment alone.

FIG. 17 is bar graph showing the increased susceptibility of biofilm-embedded E. coli treated with no antimicrobials in 50 mM citrate buffer (buffer), DspB at 20 μg/ml, cinnamaldehyde at 0.05, 0.075, and 0.1%, DspB+cinnamaldehyde at 005%, DspB+cinnamaldehyde 0.075% and DspB and cinnamaldehyde at 0.1% for 3 h at 37° C. Horizontal line was the detection limit of viable numbers. *Indicate significant reduction (P≤0.05) in viable numbers compared to buffer or DspB control. **indicate significant (P≤0.05) reduction in viable count compared to corresponding cinnamaldehyde or DspB treatment alone.

FIG. 18 is a bar graph showing biofilm dispersal activity of 50 mM citrate buffer (buffer) 20 μg/ml DspB alone and 20 μg/ml DspB plus 0.2% cinnamaldehyde. Results showed that DspB retained its activity for up to 2 h in the presence of cinnamaldehyde. *Indicate significant change (P≤0.05) in biofilm dispersal compared to DspB control.

FIG. 19 is a bar graph showing activity of DspB in the presence of 0.2% cinnamaldehyde. DspB concentration used in mixture (DspB+cinnamaldehyde) was 100 μg/mL in 50 mM citrate buffer and it's % activity was calculated compared to the activity of 100 μg/mL DspB (considered 100% activity) as a control sample. Results showed that DspB was active for up to 2 h. *Indicate significant change (P≤0.05) in DspB activity compared to DspB control.

### Example 8—Use of Octenidine Dihydrochloride

The chemical structure of octenidine dihydrochloride (OCD) is:

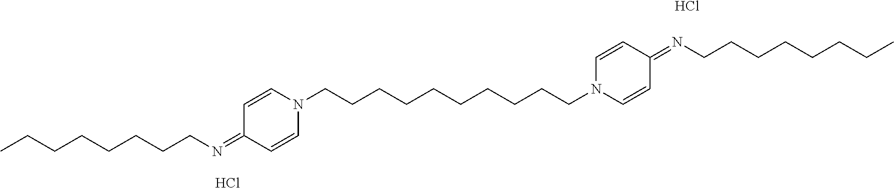 [SMILES: Cl ; CCCCCCCCN=c1ccn(CCCCCCCCCCn2ccc(=NCCCCCCCC)cc2)cc1 ; Cl]

The present invention provides uses of octenidine dihydrochloride with DspB, and compositions thereof, to enhance biofilm dispersal and inactivation of biofilm embedded bacteria. The octenidine dihydrochloride with DspB may be applied in a liquid buffer formulation, a gel formulation, or in a manner known in the art.

In an embodiment, the concentration of octenidine dihydrochloride is up to 0.15%. In a preferred embodiment, the concentration of octenidine dihydrochloride is between 0.001 and 0.1%.

In another embodiment, the concentration of DspB is up to 100 μg/ml. In a preferred embodiment, the concentration of DspB is about 200 μg/ml.

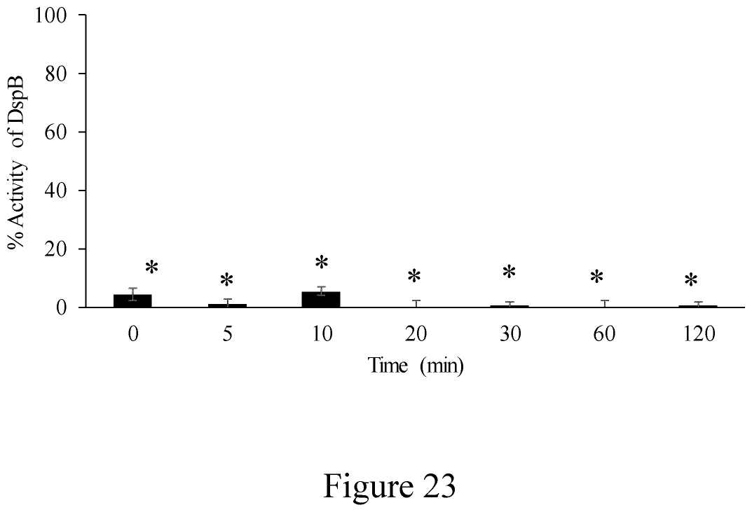

Inactivation of biofilm embedded S. epidermidis and E. coli were tested using method 1. When ODC was added with DspB, DspB enhanced (P<0.05) activity of ODC against both S. epidermidis (FIG. 20) and E. coli (FIG. 21). Compatibility between DspB and ODC was tested using biofilm dispersal assay (method 3) and enzymatic assay (method 4). Results showed that minimal biofilm dispersal was observed when DspB and ODC were added together for 2 h (FIG. 12). These results and enzymatic assay (FIG. 23) suggest that DspB lost most its activity by 2 h in the presence of ODC.

FIG. 20 is bar graph showing the increased susceptibility of biofilm-embedded S. epidermidis treated with no antimicrobials in 50 mM citrate buffer (buffer), DspB at 200 μg/ml, Octenidine dihydrochloride (ODC) at 0.001, 0.005, and 0.01%, DspB+ODC at 0.001%, DspB+ODC 0.005% and DspB and ODC at 0.01% for 10 min at 37° C. Horizontal line was the detection limit of viable numbers. *Indicate significant reduction (P≤0.05) in viable numbers compared to buffer or DspB control. **indicate significant (P≤0.05) reduction in viable count compared to corresponding ODC or DspB treatment alone.

FIG. 21 is bar graph showing the increased susceptibility of biofilm-embedded E. coli treated with no antimicrobials in 50 mM citrate buffer (buffer), DspB at 100 μg/ml, octenidine dihydrochloride (ODC) at 0.001, 0.005, and 0.01%, DspB+ODC at 0.001%, DspB+ODC 0.005% and DspB and ODC at 0.01% for 10 min at 37° C. Horizontal line was the detection limit of viable numbers. *Indicate significant reduction (P≤0.05) in viable numbers compared to buffer or DspB control. **indicate significant (P≤0.05) reduction in viable count compared to corresponding ODC or DspB treatment alone.

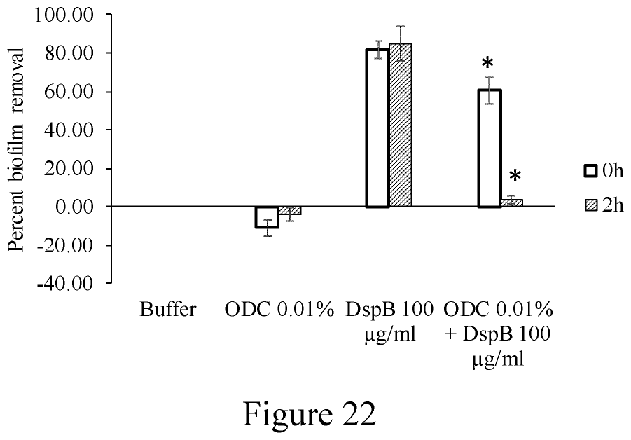

FIG. 22 is a bar graph showing biofilm dispersal activity of, 50 mM citrate buffer (buffer), 0.01% ODC alone, 100 μg/ml DspB alone and 100 μg/ml DspB plus 0.01% octenidine dihydrochloride (ODC). Results showed that DspB lost its activity by 2 h in the presence of ODC. *Indicate significant reduction (P≤0.05) in biofilm dispersal compared to DspB control.

FIG. 23 is a bar graph showing activity of DspB in the presence of 0.1% octenidine dihydrochloride (ODC). DspB concentration used in mixture (DspB+ODC) was 100 μg/mL in 50 mM citrate buffer and its % activity was calculated compared to the activity of 100 μg/mL DspB (considered 100% activity) as a control sample. Results showed that DspB was active for up to 2 h. *Indicate significant change (P≤0.05) in DspB activity compared to DspB control.

### Example 9—Use of Cetylpyridinium Chloride (CPC)

The chemical structure of Cetylpyridinium chloride is:

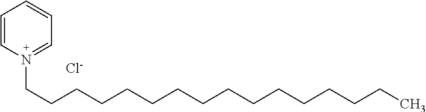

The present invention provides uses of CPC with DspB, and compositions thereof, to enhance biofilm dispersal and inactivation of biofilm embedded bacteria. The CPC with DspB may be applied in a liquid buffer formulation, a gel formulation, or in a manner known in the art.

Biofilm dispersal assay indicate DspB was active immediately after mixing with CPC and it lost its activity by 2 h in the presence of CPC.

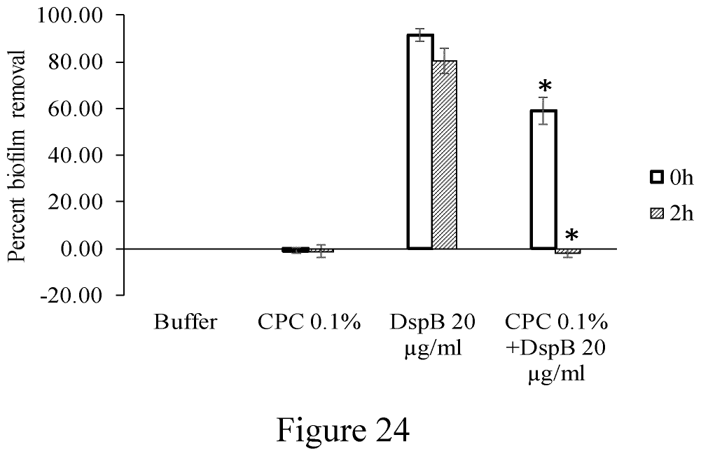

FIG. 24 is a bar graph showing biofilm dispersal activity of, 50 mM phosphate buffer (buffer), 0.1% cetylpyridinium chloride (CPC) alone, 20 μg/ml DspB alone and 20 μg/ml DspB plus 0.1% CPC. Results showed that DspB lost its activity by 2 h in the presence of CPC. *Indicate significant reduction (P≤0.05) in biofilm dispersal compared to DspB control.

### Example 10—Use of Providone Iodine

The chemical structure of providone iodine is:

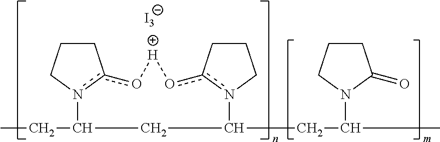

The present invention provides uses of providone iodine with DspB, and compositions thereof, to enhance biofilm dispersal and inactivation of biofilm embedded bacteria. The providone iodine with DspB may be applied in a liquid buffer formulation, a gel formulation, or in a manner known in the art.

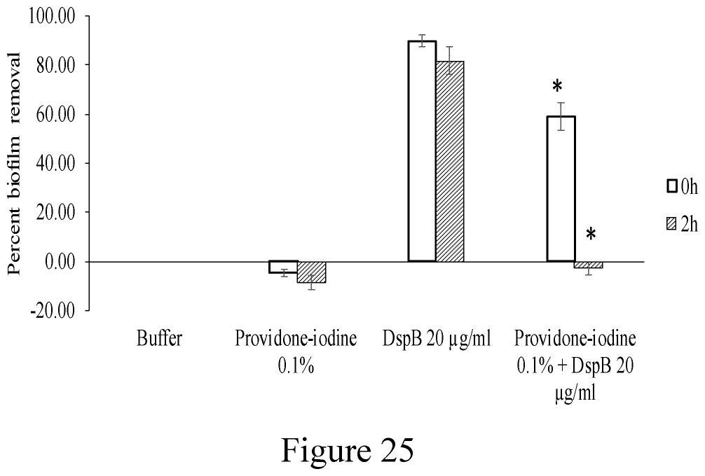

Data (FIG. 25) showed that DspB was active immediately after mixing with providone iodine and lost its activity by 2 h in the presence of providone iodine.

FIG. 25 is a bar graph showing biofilm dispersal activity of, 50 mM phosphate buffer (buffer), 20 μg/ml DspB alone and 20 μg/ml DspB plus 0.1% active iodine (1% providone iodine. Results showed that DspB lost its activity by 2 h in the presence of iodine. *Indicate significant reduction (P≤0.05) in biofilm dispersal compared to DspB control.

### Example 11—Use of Gentamicin Sulfate

The chemical structure of Gentamicin sulfate is:

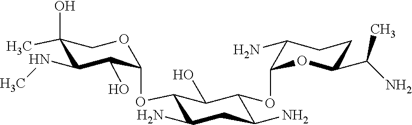

The present invention provides uses of Gentamicin sulfate with DspB, and compositions thereof, to enhance biofilm dispersal and inactivation of biofilm embedded bacteria. The Gentamicin sulfate with DspB may be applied in a liquid buffer formulation, a gel formulation, or in a manner known in the art.

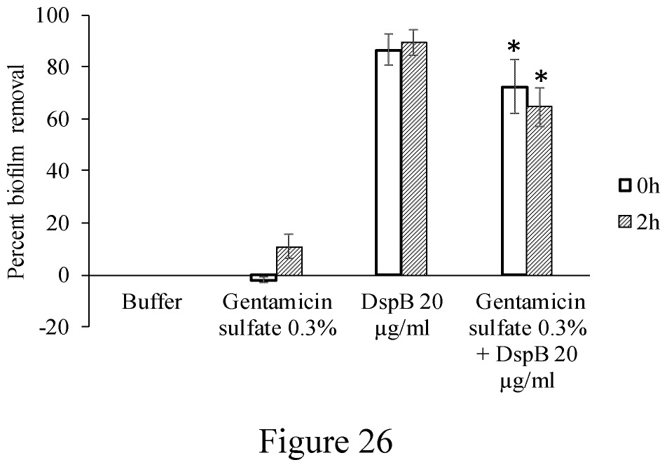

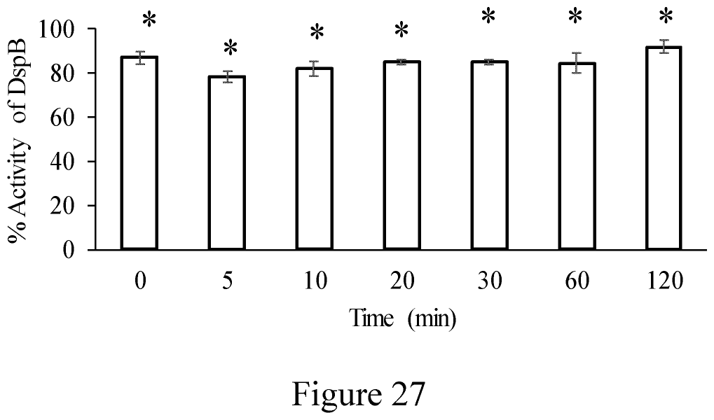

Based on biofilm dispersal assay (FIG. 26) and enzymatic assay (FIG. 27), DspB remained active for up to 2 h in the presence of Gentamicin sulfate.

FIG. 26 is a bar graph showing biofilm dispersal activity of, 50 mM phosphate buffer (buffer), 0.3% gentamicin sulfate, 20 μg/ml DspB alone and 20 μg/ml DspB plus 0.3% gentamicin sulfate. Results showed that DspB was active for upto 2 h in the presence of gentamicin sulfate. *Indicate significant reduction (P≤0.05) in biofilm dispersal compared to DspB control.

FIG. 27 is a bar graph showing activity of DspB in the presence of 0.3% Gentamicin sulfate. DspB concentration used in mixture (DspB+Gentamicin sulfate) was 100 μg/mL in 50 mM phosphate buffer and it's % activity was calculated compared to the activity of 100 μg/mL DspB (considered 100% activity) as a control sample. Results showed that DspB was active in the presence of Gentamicin sulfate for up to 2 h. *Indicate significant reduction (P≤0.05) in DspB activity compared to DspB control.

### Example 12—Use of Neomycin Sulphate

The chemical structure of Neomycin sulphate is:

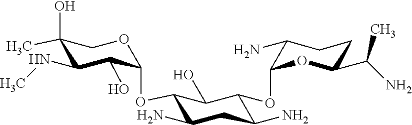

The present invention provides uses of Neomycin sulphate with DspB, and compositions thereof, to enhance biofilm dispersal and inactivation of biofilm embedded bacteria. The Neomycin sulphate with DspB may be applied in a liquid buffer formulation, a gel formulation, or in a manner known in the art.

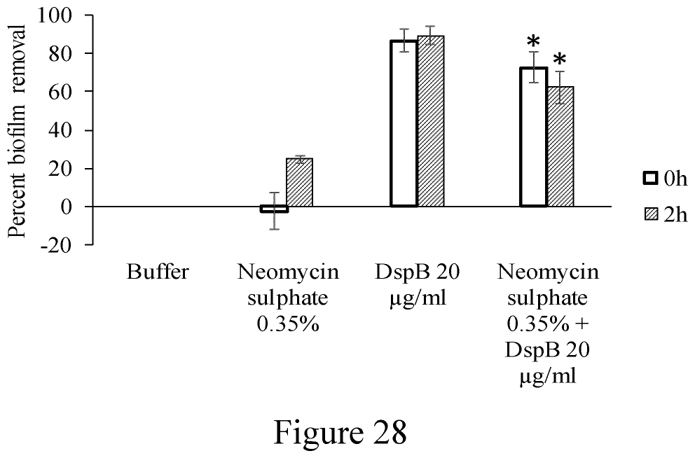

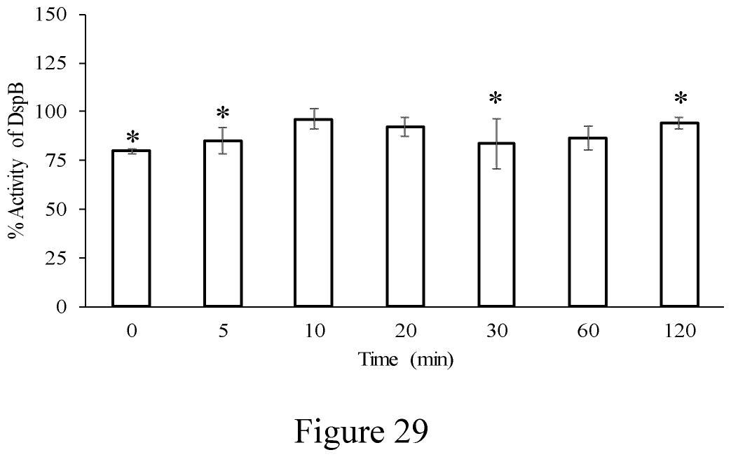

Based on biofilm dispersal assay (FIG. 28) and enzymatic assay (FIG. 29), DspB remained active for up to 2 h in the presence of Neomycin sulphate.

FIG. 28 is a bar graph showing biofilm dispersal activity of, 50 mM phosphate buffer (buffer), 0.35% Neomycin sulphate alone, 20 μg/ml DspB alone and 20 μg/ml DspB plus 0.35% Neomycin sulphate. Results showed that DspB was active for up to 2 h in the presence of Neomycin sulphate. *Indicate significant reduction (P≤0.05) in biofilm dispersal compared to DspB control.

FIG. 29 is a bar graph showing activity of DspB in the presence of 0.3% Gentamicin sulfate. DspB concentration used in mixture (DspB+Neomycin sulphate) was 100 μg/mL in 50 mM phosphate buffer and it's percent activity was calculated compared to the activity of 100 μg/mL DspB (considered 100% activity) as a control sample. Results showed that DspB was active in the presence of Neomycin sulphate for up to 2 h. *Indicate significant reduction (P≤0.05) in DspB activity compared to DspB control.

### Example 13—Use of Polymyxin B Sulfate

The chemical structure of Polymyxin B sulfate is:

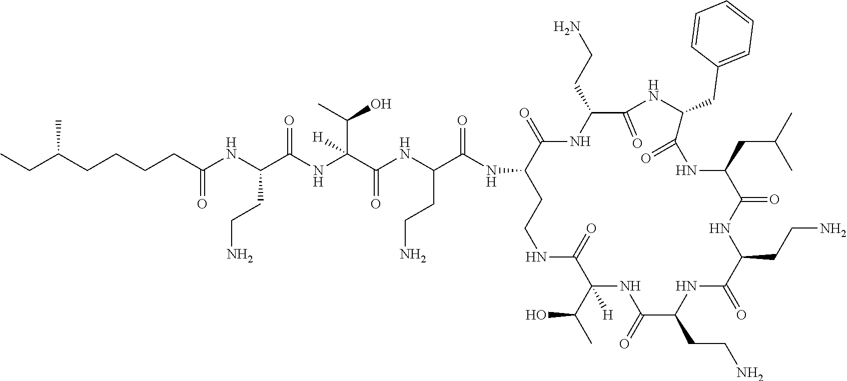 [SMILES: CC[C@H](C)CCCCC(=O)N[C@@H](CCN)C(=O)N[C@H](C(=O)NC(CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@@H](CCN)NC1=O)[C@@H](C)O]

The present invention provides uses of Polymyxin B sulfate with DspB, and compositions thereof, to enhance biofilm dispersal and inactivation of biofilm embedded bacteria. The Polymyxin B sulfate with DspB may be applied in a liquid buffer formulation, a gel formulation, or in a manner known in the art.

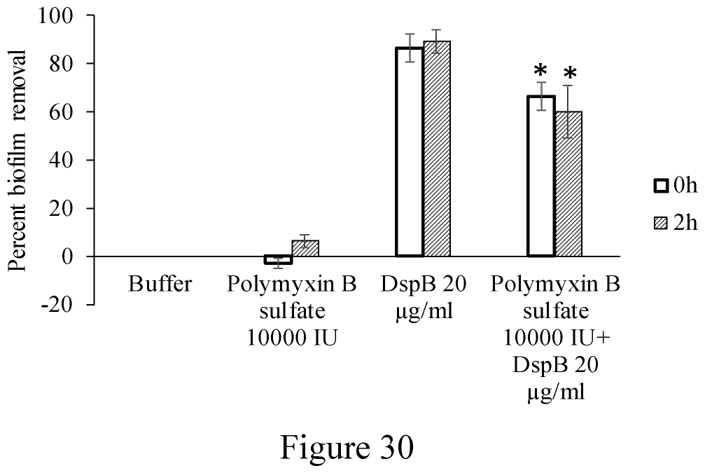

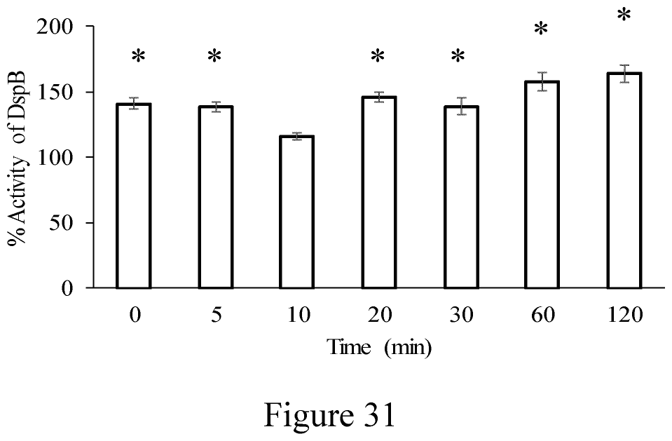

Based on biofilm dispersal assay (FIG. 30) and enzymatic assay (FIG. 31), DspB remained active for up to 2 h in the presence of Polymyxin B sulfate.

FIG. 30 is a bar graph showing biofilm dispersal activity of, 50 mM phosphate buffer (buffer), 10000 IU polymyxin B sulfate alone, 20 μg/ml DspB alone and 20 μg/ml DspB plus 10,000 IU (0.167%) Polymyxin B sulfate. Results showed that DspB was active for up to 2 h in the presence of Polymyxin B sulfate. *Indicate significant reduction (P≤0.05) in biofilm dispersal compared to DspB control.

FIG. 31 is a bar graph showing activity of DspB in the presence of 5000 IU (0.083%) Polymyxin B sulfate. DspB concentration used in mixture (DspB+Polymyxin B sulfate) was 100 μg/mL in 50 mM phosphate buffer and it's percent activity was calculated compared to the activity of 100 μg/mL DspB (considered 100% activity) as a control sample. Results showed that DspB was active in the presence of Polymyxin B sulfate for up to 2 h. *Indicate significant increase (P≤0.05) in DspB activity compared to DspB control.

### Example 14—Use of Mupirocin

The chemical structure of Mupirocin

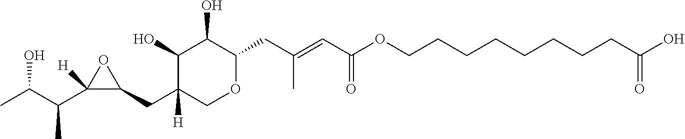 [SMILES: C/C(=C\C(=O)OCCCCCCCCC(=O)O)C[C@@H]1OC[C@H](C[C@@H]2O[C@H]2[C@@H](C)[C@H](C)O)[C@@H](O)[C@H]1O]

The present invention provides uses of Mupirocin with DspB, and compositions thereof, to enhance biofilm dispersal and inactivation of biofilm embedded bacteria. The Mupirocin with DspB may be applied in a liquid buffer formulation, a gel formulation, or in a manner known in the art.

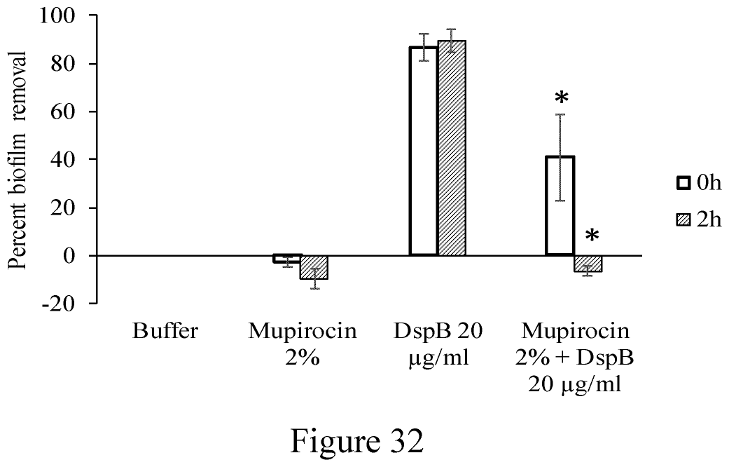

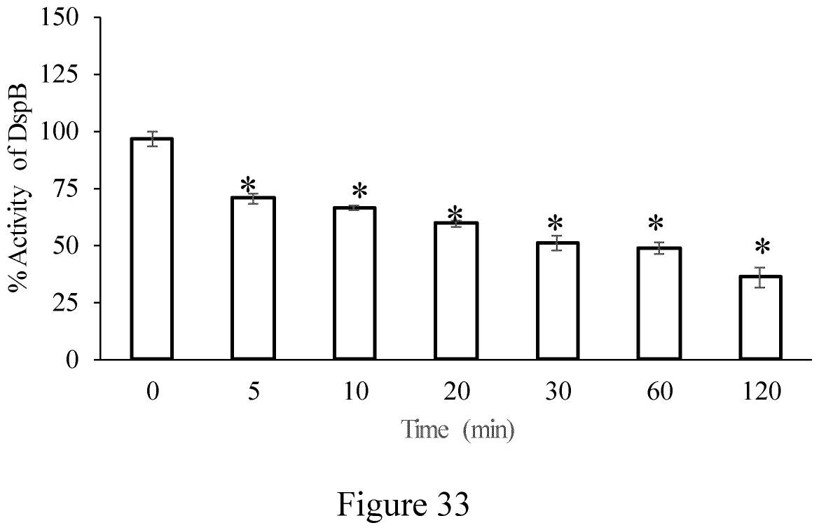

Based on biofilm dispersal assay (FIG. 32) DspB was active at 0 h and lost its activity by 2 h. However, based on enzymatic assay (FIG. 33), DspB remained active for up to 2 h in the presence of Mupirocin.

FIG. 32 is a bar graph showing biofilm dispersal activity of, 50 mM phosphate buffer (buffer), 20 μg/ml DspB alone and 20 μg/ml DspB plus 2% Mupirocin. Results showed that DspB was inactivated by 2 h in the presence of Mupirocin. *Indicate significant reduction (P≤0.05) in biofilm dispersal compared to DspB control.

FIG. 33 is a bar graph showing activity of DspB in the presence of 2% Mupirocin. DspB concentration used in mixture (DspB+Mupirocin) was 100 μg/mL in 50 mM phosphate buffer and it's percent activity was calculated compared to the activity of 100 μg/mL DspB (considered 100% activity) as a control sample. Results showed that DspB was active in the presence of Mupirocin for up to 2 h. *Indicate significant increase (P≤0.05) in DspB activity compared to DspB control.

### Example 15—Use of Gramicidin

Gramicidin is a mix of ionophoric antibiotics.

The present invention provides uses of Gramicidin with DspB, and compositions thereof, to enhance biofilm dispersal and inactivation of biofilm embedded bacteria. The Gramicidin with DspB may be applied in a liquid buffer formulation, a gel formulation, or in a manner known in the art.

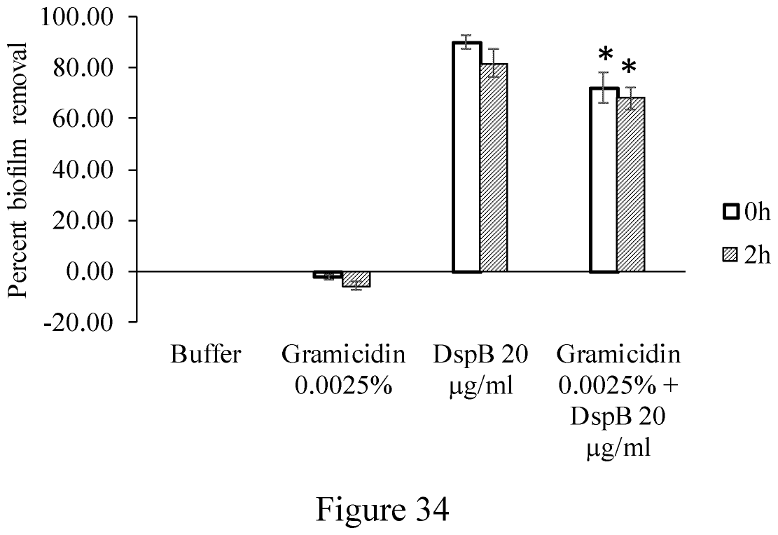

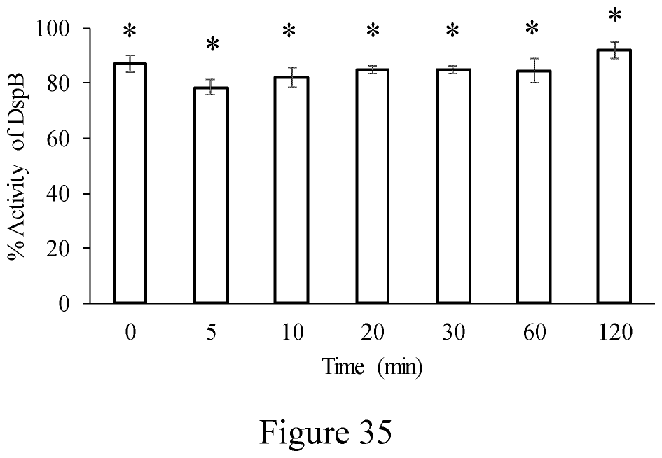

Based on biofilm dispersal assay (FIG. 34) and enzymatic assay (FIG. 35), DspB remained active for up to 2 h in the presence of Gramicidin.

FIG. 34 is a bar graph showing biofilm dispersal activity of, 50 mM phosphate buffer (buffer), 0.0025% gramicidin alone, 20 μg/ml DspB alone and 20 μg/ml DspB plus 0.0025% gramicidin. Results showed that DspB was active for up to 2 h in the presense of gramicidin. *Indicate significant reduction (P≤0.05) in biofilm dispersal compared to DspB control.

FIG. 35 is a bar graph showing activity of DspB in the presence of 0.0025% Gramicidin. DspB concentration used in mixture (DspB+Gramicidin) was 100 μg/mL in 50 mM phosphate buffer and it's percent activity was calculated compared to the activity of 100 μg/mL DspB (considered 100% activity) as a control sample. Results showed that DspB was active in the presence of gramicidin for up to 2 h. *Indicate significant increase (P≤0.05) in DspB activity compared to DspB control.

### Example 16—Use of Metronidazole

The chemical structure of Metronidazole is:

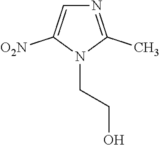

The present invention provides uses of Metronidazole with DspB, and compositions thereof, to enhance biofilm dispersal and inactivation of biofilm embedded bacteria. The Metronidazole with DspB may be applied in a liquid buffer formulation, a gel formulation, or in a manner known in the art.

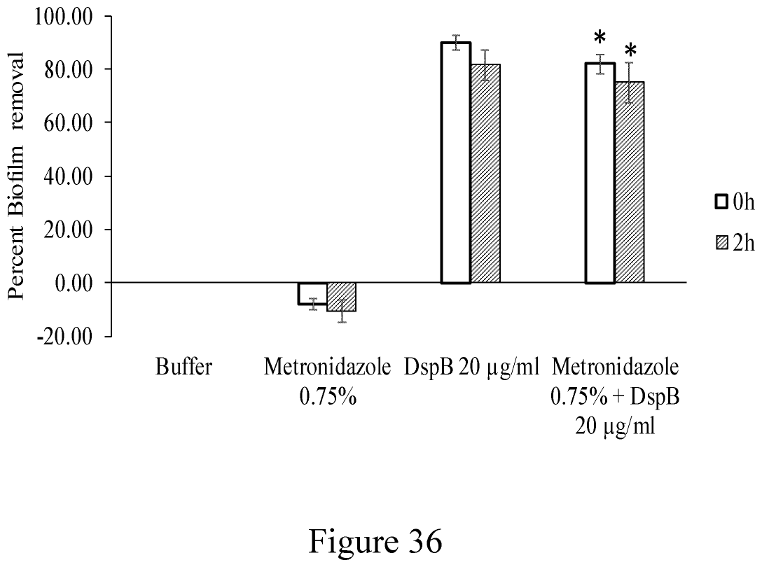

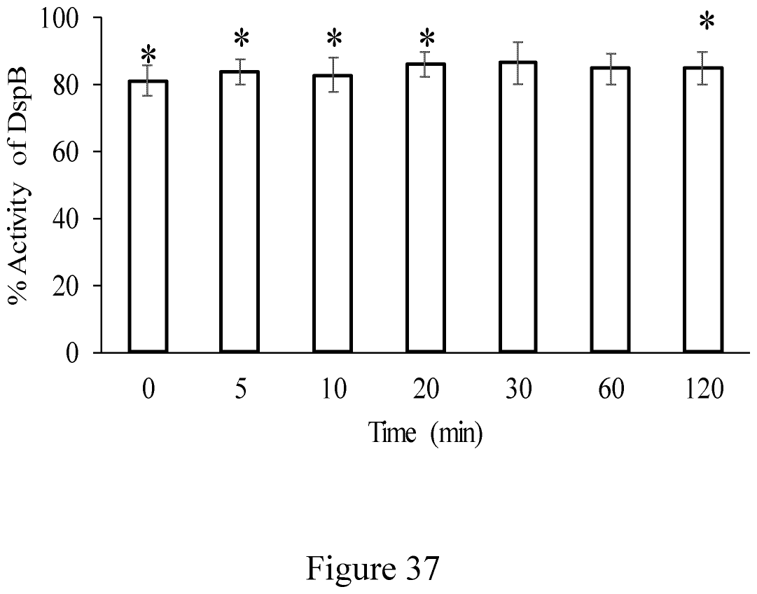

Based on biofilm dispersal assay (FIG. 36) and enzymatic assay (FIG. 37), DspB remained active for up to 2 h in the presence of Metronidazole.

FIG. 36 is a bar graph showing biofilm dispersal activity of, 50 mM phosphate buffer (buffer), 0.75% metronidazole alone, 20 μg/ml DspB alone and 20 μg/ml DspB plus 0.75% Metronidazole. Results showed that DspB was active for up to 2 h in the presence of Metronidazole. *Indicate significant reduction (P≤0.05) in biofilm dispersal compared to DspB control.

FIG. 37 is a bar graph showing activity of DspB in the presence of 0.75% Metronidazole. DspB concentration used in mixture (DspB+Metronidazole) was 100 μg/mL in 50 mM phosphate buffer and it's percent activity was calculated compared to the activity of 100 μg/mL DspB (considered 100% activity) as a control sample. Results showed that DspB was active in the presence of Metronidazole for up to 2 h. *Indicate significant increase (P≤0.05) in DspB activity compared to DspB control.

### Wound Gel Formulations

### Example 17—Wound Gel With PAPB

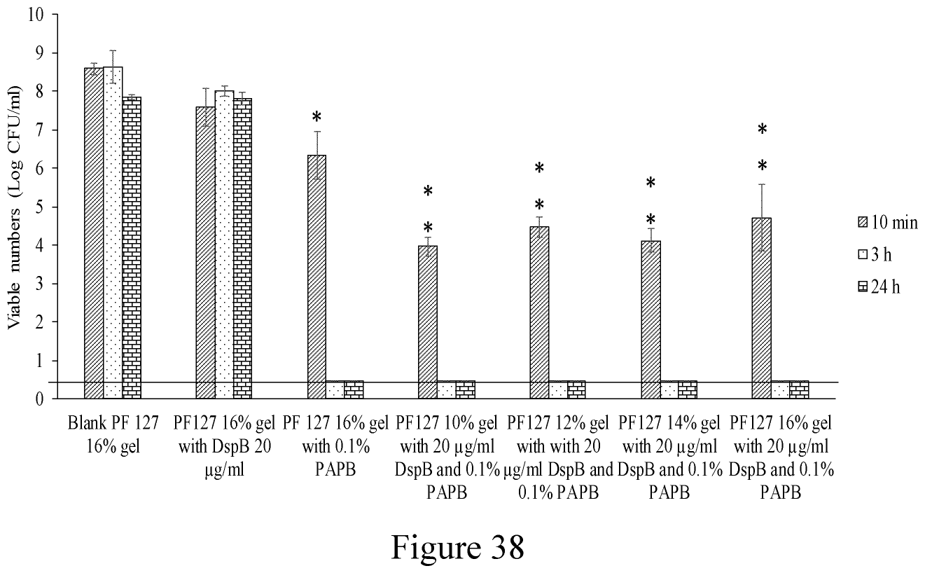

FIG. 38 is bar graph showing the increased susceptibility of biofilm-embedded S. epidermidis treated with no antimicrobials (Blank Poloxamer 405; PF 127 16% gel in 50 mM phosphate buffer; P-buffer), PF 127 16% gel with DspB at 20 μg/ml, PF 127 16% gel with 0.1% PAPB, PF 127 10% gel with 20 μg/ml DspB plus 0.1% PAPB, PF 127 12% gel with 20 μg/ml DspB plus 0.1% PAPB, PF 127 14% gel with 20 μg/ml DspB plus 0.1% PAPB and PF 127 16% gel with 20 μg/ml DspB plus 0.1% PAPB. Horizontal line was the detection limit of viable numbers. *Indicate significant reduction (P≤0.05) in viable numbers compared to blank gel or gel with DspB alone. **indicate significant (P≤0.05) reduction in viable count compared to treatment with gel containing DspB alone or gel containing PAPB alone.

### Example 18—Wound Gel With PAPB and PHMB

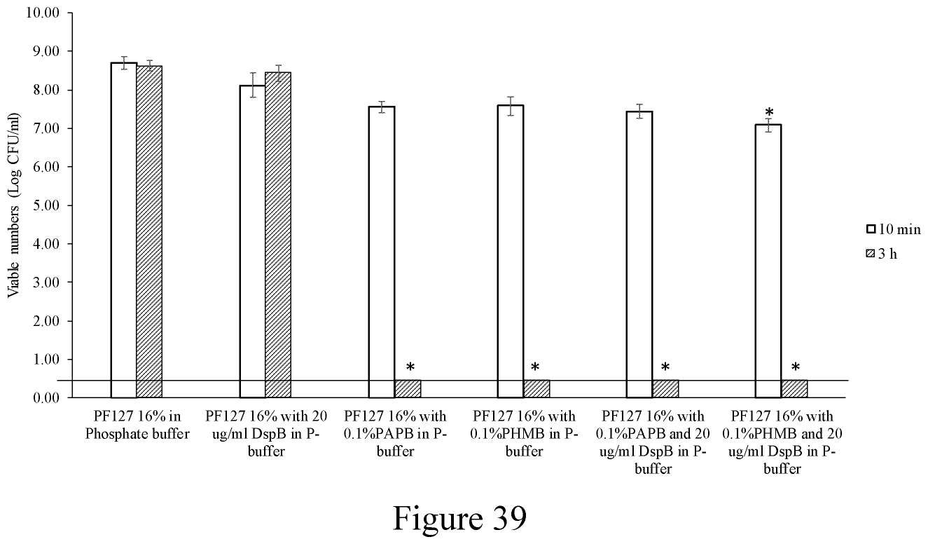

FIG. 39 is bar graph showing the increased susceptibility of biofilm-embedded S. epidermidis treated with Blank PF 127 16% gel in 50 mM phosphate buffer (P-buffer), PF 127 16% gel with DspB at 20 μg/ml in P-buffer, PF 127 16% gel with 0.1% PAPB in P-buffer, PF 127 16% gel with 0.1% PHMB in P-buffer, PF 127 16% gel with 20 μg/ml DspB plus 0.1% PAPB in P-buffer and PF 127 16% gel with 20 μg/ml DspB plus 0.1% PHMB in P-buffer. Horizontal line was the detection limit of viable numbers. *Indicate significant reduction (P≤0.05) in viable numbers compared to blank gel, gel with DspB alone, or gel containing PAPB/PHMB alone.

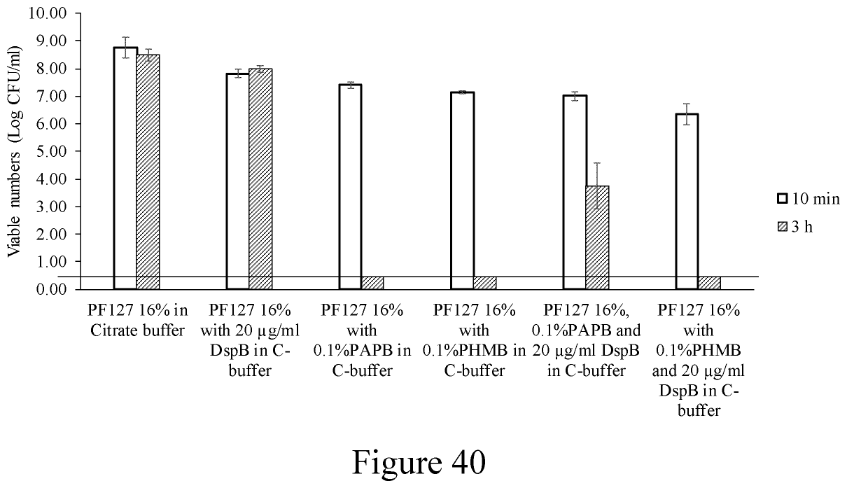

FIG. 40 is bar graph showing the increased susceptibility of biofilm-embedded S. epidermidis treated with Blank PF 127 16% gel in 50 mM citrate buffer (C-buffer), PF 127 16% gel with DspB at 20 μg/ml in C-buffer, PF 127 16% gel with 0.1% PAPB in C-buffer, PF 127 16% gel with 0.1% PHMB in C-buffer, PF 127 16% gel with 20 μg/ml DspB plus 0.1% PAPB in C-buffer and PF 127 16% gel with 20 μg/ml DspB plus 0.1% PHMB in C-buffer. Horizontal line was the detection limit of viable numbers. *Indicate significant reduction (P≤0.05) in viable numbers compared to blank gel, gel with DspB alone, or gel with PAPB or PHMB alone.

### Example 19—Wound Gel With PHMB

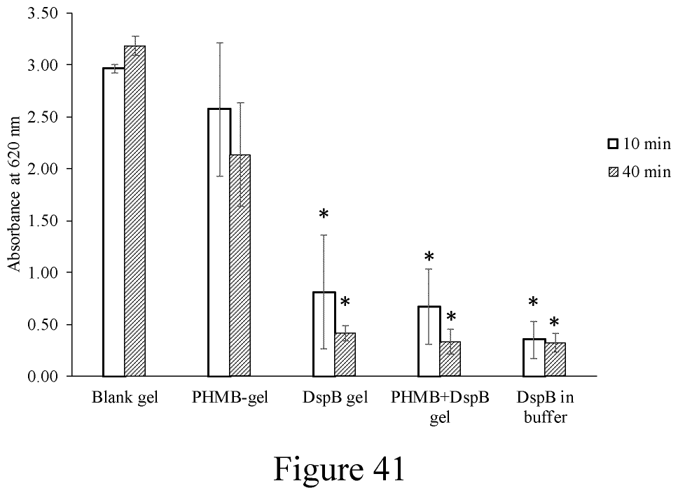

FIG. 41 is bar graph showing dispersal of S. epidermidis biofilm treated with Blank PF 127 16% gel in 50 mM phosphate buffer (P-buffer), PF 127 16% gel with DspB at 20 μg/ml in P-buffer, PF 127 16% gel with 0.1% PHMB in P-buffer, PF 127 16% gel with 20 μg/ml DspB plus 0.1% PHMB in P-buffer and 20 μg/ml DspB in phosphate buffer (positive control). *Indicate significant (P≤0.05) biofilm dispersal compared to blank gel or gel with PHMB alone after 10 or 40 min treatment.

### Example 20—Stability of PHMB and PAPB in Wound Gel Formulation

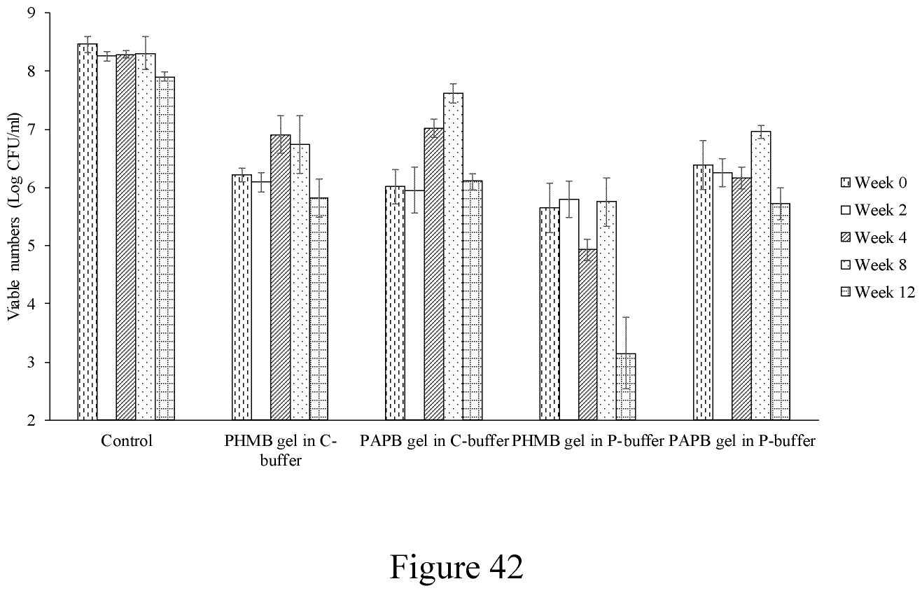

FIG. 42 is bar graph showing inactivation of biofilm embedded S. epidermidis by wound gel formulations containing PF 127 16% gel in citrate buffer (C-buffer) containing DspB at 20 μg/ml plus 0.1% PHMB, PF 127 16% gel in C-buffer containing DspB at 20 μg/ml plus 0.1% PAPB, PF 127 16% gel in phosphate buffer (P-buffer) containing DspB at 20 μg/ml plus 0.1% PHMB, PF 127 16% gel in P-buffer containing DspB at 20 μg/ml plus 0.1% PAPB and water control. Wound gel formulation was stored at 45° C. for 12 weeks, which is equivalent to one-year shelf-life at room temperature (20-25° C.). Wound gel samples were taken at pre-determined intervals and tested for their ability to inactivate biofilm embedded S. epidermidis using method 2. Biofilms developed in 96-microwell plates were tested using gels for 45 min at 37° C. Then washed and viable numbers were determined by plating on TSA agar. Results showed that wound gel formulations retained their anti-biofilm activity for 12 weeks, which is equivalent to one-year shelf-life at room temperature (20-25° C.).

TABLE 3
Antimicrobial activity of wound gel formulations after storing at 45° C. for 12 weeks determined
using disc diffusion assay. All wound gel types retained their antimicrobial activity for 12
weeks at 45° C., which is equivalent to one-year shelf-life at room temperature (20-25° C.).

| | Zone of inhibition | |
|---|---|---|
| | diameter (mm) | |
| Wound gel type | Week 0 | Week 12 |
| PF 127 16% gel in citrate buffer(C-buffer) containing DspB at 20 μg/ml | 23 ± 1.0 | 21.33 ± 0.6 |
| plus 0.1% PHMB | | |
| PF 127 16% gel in C-buffer containing DspB at 20 μg/ml plus 0.1% PAPB | 21.67 ± 1.7 | 21.67 ± 0.6 |
| PF 127 16% gel in phosphate buffer (P-buffer) containing DspB at 20 | 24.33 ± 0.6 | 20.67 ± 0.6 |
| μg/ml plus 0.1% PHMB, | | |
| PF 127 16% gel in P-buffer containing DspB at 20 μg/ml plus 0.1% PAPB | 22.0 ± 1.0 | 19.0 ± 1.0 |

### Example 21—Stability of DspB in Gel Formulations in the Presence of Antimicrobials and Antibiotics

Gels containing DspB and selected concentrations of antimicrobials were formulated in 50 mM phosphate buffer or citrate buffer containing 16% PF127. DspB gel and antimicrobial gel were mixed together and incubated at 37° C. for 2 h. Biofilm dispersal was performed at 0 and 2 h using biofilm dispersal assay (Method 5). Blank gel, DspB gel and antimicrobial gels were included as controls.

Stability of DspB gels also assed using enzymatic assay (Method 6).

### Example 22—PHMB and DspB in Gel

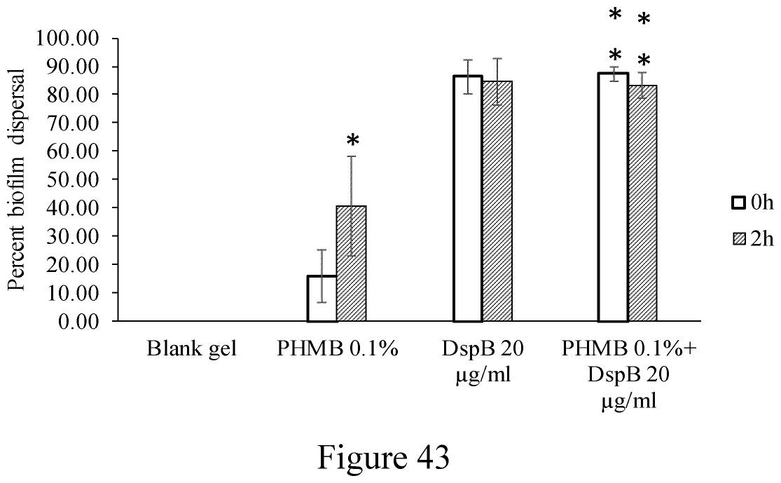

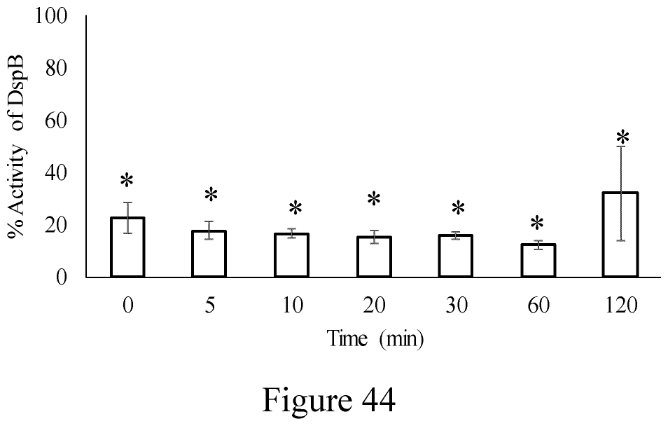

Based on biofilm dispersal assay (FIG. 43) and enzymatic assay (FIG. 44), DspB remained active for up to 2 h in the presence of PHMB in wound gel.

FIG. 43 is a bar graph showing biofilm dispersal activity of PF127 gel prepared in 50 mM phosphate buffer (Blank gel), 0.1% PHMB alone, 20 μg/ml DspB alone and 20 μg/ml DspB plus 20 μg/ml PHMB. Results showed that DspB retained its activity for up to 2 h in the presence of PHMB. Biofilm dispersal by DspB gel and PHMB 0.1% plus DspB gel were similar (P>0.05). However, biofilm Dispersal by gel containing PHMB 0.1% plus DspB was significantly higher than the gel containing PHMB alone (P≤0.05).

FIG. 44 is a bar graph showing activity of DspB in the presence of 0.1% PHMB. DspB concentration used in mixture (DspB+PHMB) was 100 μg/mL in PF 127 gel prepared in 50 mM phosphate buffer and it's % activity was calculated compared to the activity of 100 μg/mL DspB (considered 100% activity) as a control sample. Results showed that DspB was active for up to 2 h. *Indicate significant reduction (P≤0.05) in DspB activity compared to DspB control.

### Example 23—Carvacrol and DspB Gel

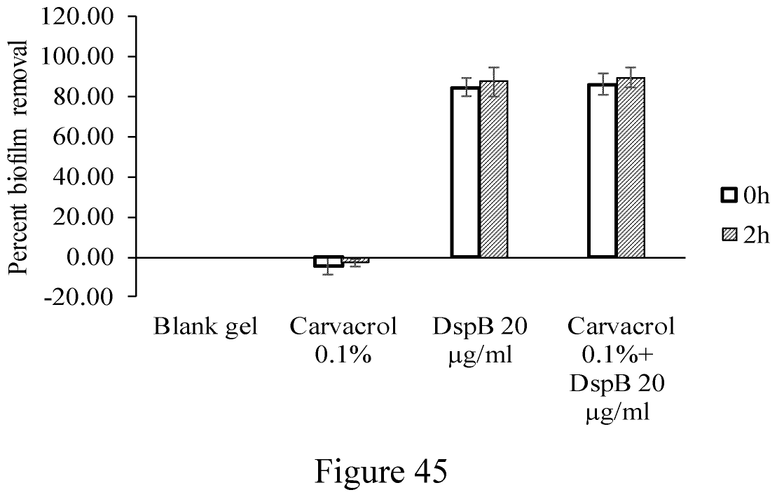

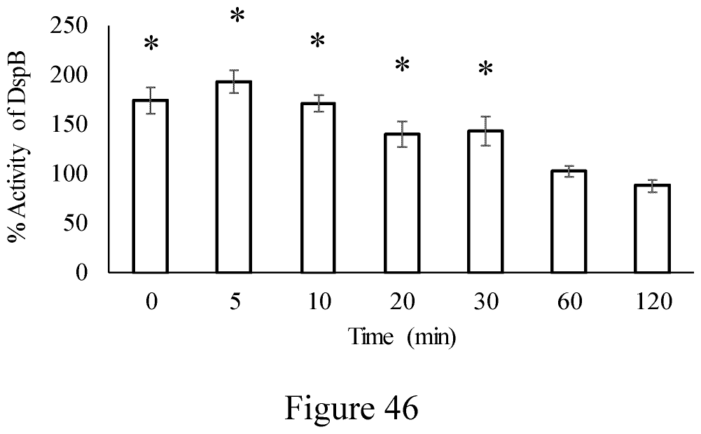

Based on biofilm dispersal assay (FIG. 45) and enzymatic assay (FIG. 46), DspB remained active for up to 2 h in the presence of carvacrol in wound gel. Higher enzymatic activity (P≤0.05) was observed in the presence of carvacrol (FIG. 46) at 0-30 min.

FIG. 45 is a bar graph showing biofilm dispersal activity of PF127 gel prepared in 50 mM citrate buffer (Blank gel), 0.1% carvacrol alone, 20 μg/ml DspB alone and 20 μg/ml DspB plus 0.1% carvacrol. Results showed that DspB retained its activity for up to 2 h in the presence of carvacrol. Biofilm dispersal activity of gel containing DspB alone was similar to the gel containing DspB and carvacrol (P>0.05).

FIG. 46 is a bar graph showing activity of DspB in the presence of 0.1% carvacrol. DspB concentration used in mixture (DspB+carvacrol) was 100 μg/mL in PF127 gel prepared in 50 mM citrate buffer and it's % activity was calculated compared to the activity of 100 μg/mL DspB (considered 100% activity) as a control sample. Results showed that DspB was active for up to 2 h. *Indicate significant increase (P≤0.05) in DspB activity compared to DspB control.

### Example 24—Chloroxylenol and DspB Gel

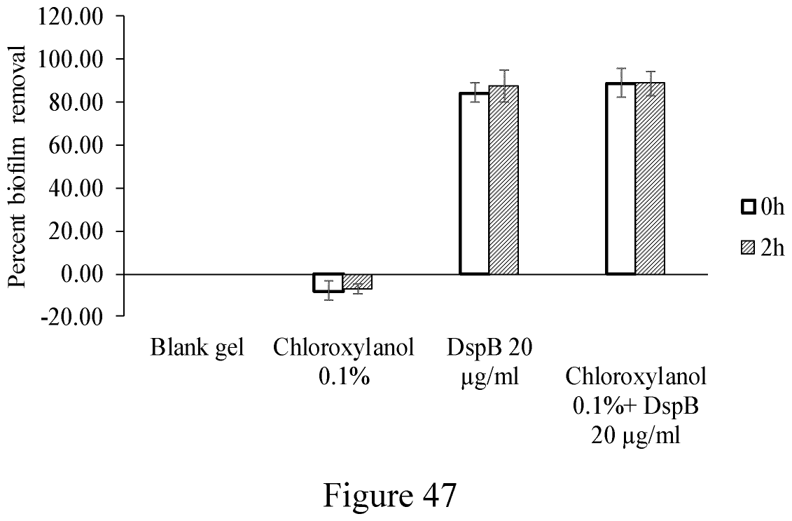

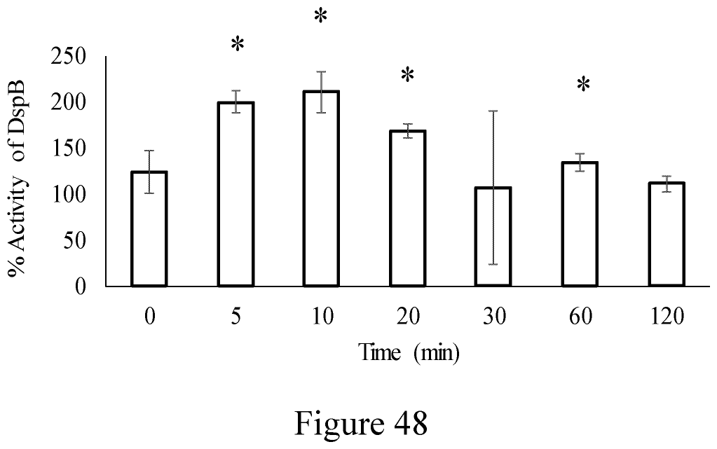

Based on biofilm dispersal assay (FIG. 47) and enzymatic assay (FIG. 48), DspB remained active for up to 2 h in the presence of chloroxylenol in wound gel. Higher enzymatic activity (P≤0.05) was observed in the presence of chloroxylenol (FIG. 46) at 5, 10, 20 and 60 min.

FIG. 47 is a bar graph showing biofilm dispersal activity of PF127 gel prepared in 50 mM citrate buffer (buffer), 0.1% chloroxylenol alone, 20 μg/ml DspB alone and 20 μg/ml DspB plus 0.1% chloroxylenol. Results showed that DspB retained its activity for up to 2 h in the presence of chloroxylenol. Biofilm dispersal activity of gel containing DspB alone was similar to the gel containing DspB and carvacrol (P>0.05).

FIG. 48 is a bar graph showing activity of DspB in the presence of 0.1% chloroxylenol. DspB concentration used in mixture (DspB+chloroxylenol) was 100 μg/mL in PF127 gel prepared in 50 mM citrate buffer and it's % activity was calculated compared to the activity of 100 μg/mL DspB (considered 100% activity) as a control sample. Results showed that DspB was active for up to 2 h. *Indicate significant increase (P≤0.05) in DspB activity compared to DspB control.

### Example 25—Cinnamaldehyde and DspB Gel

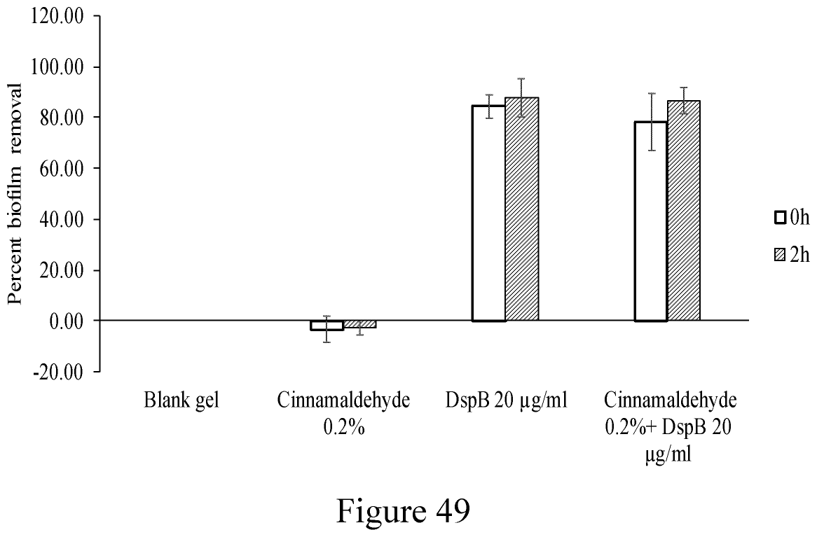

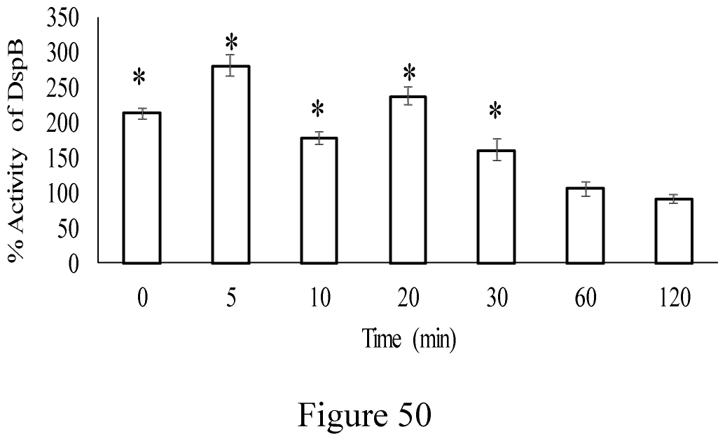

Based on biofilm dispersal assay (FIG. 49) and enzymatic assay (FIG. 50), DspB remained active for up to 2 h in the presence of cinnamaldehyde in wound gel. Higher enzymatic activity (P≤0.05) was observed in the presence of cinnamaldehyde (FIG. 50) at 0-30 min.

FIG. 49 is a bar graph showing biofilm dispersal activity of PF127 gel prepared in 50 mM citrate buffer (buffer), 0.1% cinnamaldehyde alone, 20 μg/ml DspB alone and 20 μg/ml DspB plus 0.1% cinnamaldehyde. Results showed that DspB retained its activity for up to 2 h in the presence of cinnamaldehyde. Biofilm dispersal activity of gel containing DspB alone was similar to the gel containing DspB and cinnamaldehyde (P>0.05).

FIG. 50 is a bar graph showing activity of DspB in the presence of 0.1% cinnamaldehyde. DspB concentration used in mixture (DspB+cinnamaldehyde) was 100 μg/mL in PF127 gel prepared in 50 mM citrate buffer and it's % activity was calculated compared to the activity of 100 μg/mL DspB (considered 100% activity) as a control sample. Results showed that DspB was active for up to 2 h. *Indicate significant increase (P≤0.05) in DspB activity compared to DspB control.

### Example 26—Octenidine Dihydrochloride (ODC)

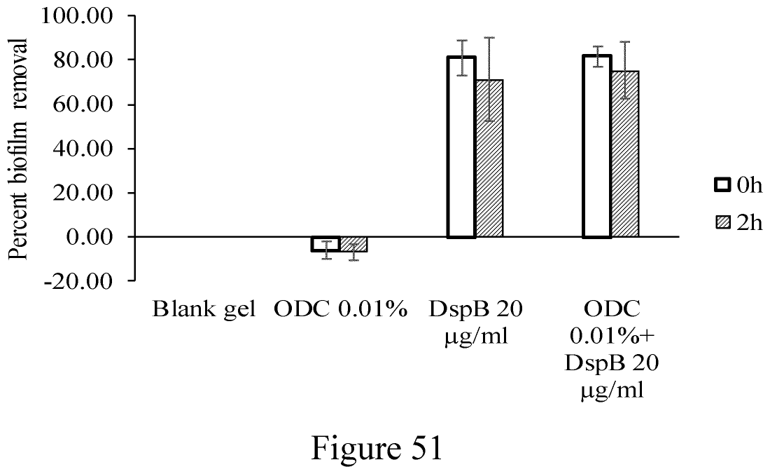

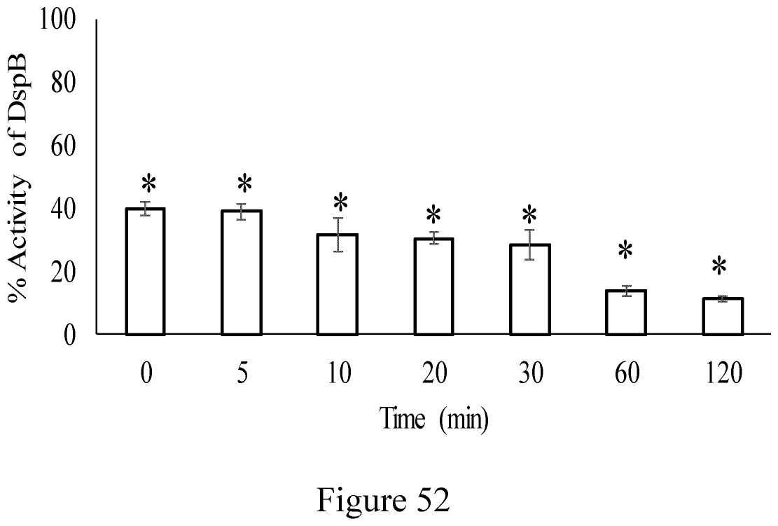

Based on biofilm dispersal assay (FIG. 51) and enzymatic assay (FIG. 52), DspB remained active for up to 2 h in the presence of ODC in wound gel. In contrast to results observed in buffer formulation (FIG. 22), where DspB activity was lost by 2 h in the presence of ODC, in gel formulation DspB remained active for 2 h and biofilm dispersal was similar to biofilm dispersal by gel containing DspB alone.

FIG. 51 is a bar graph showing biofilm dispersal activity of PF127 gel prepared in 50 mM citrate buffer (buffer), 0.01% ODC alone, 20 μg/ml DspB alone and 20 μg/ml DspB plus 0.1% ODC. Results showed that DspB retained its activity for up to 2 h in the presence of cinnamaldehyde. Biofilm dispersal activity of gel containing DspB alone was similar to the gel containing DspB and ODC (P>0.05).

FIG. 52 is a bar graph showing activity of DspB in the presence of 0.1% ODC. DspB concentration used in mixture (DspB+ODC) was 100 μg/mL in PF127 gel prepared in 50 mM citrate buffer and it's % activity was calculated compared to the activity of 100 μg/mL DspB (considered 100% activity) as a control sample. Results showed that DspB was active for up to 2 h. *Indicate significant change (P≤0.05) in DspB activity compared to DspB control.

### Example 27—Cetylpyridinium Chloride (CPC) and DspB Gel

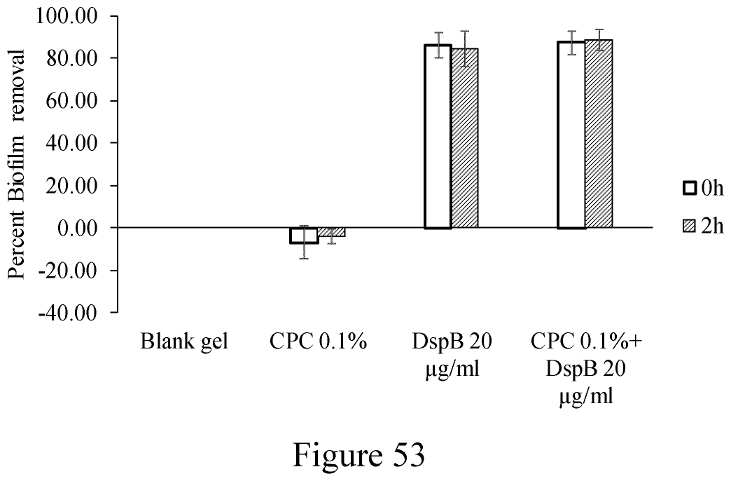

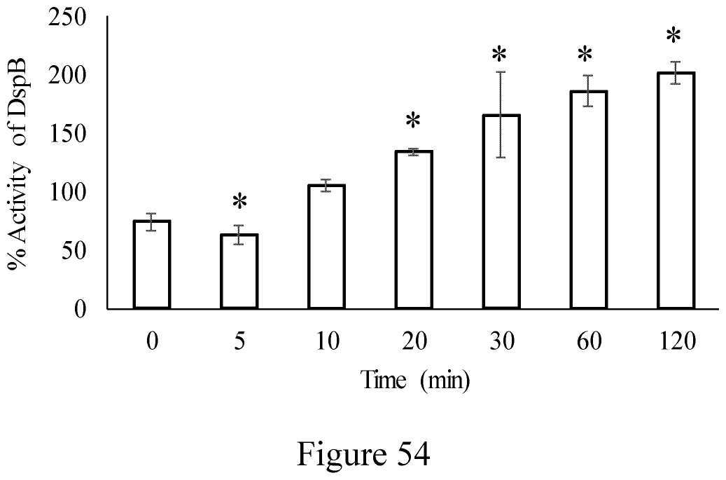

Based on biofilm dispersal assay (FIG. 53) and enzymatic assay (FIG. 54), DspB remained active for up to 2 h in the presence of CPC in wound gel. In contrast to results observed in buffer formulation (FIG. 24), where DspB activity was lost by 2 h in the presence of CPC, DspB remained active for 2 h in gel formulation and biofilm dispersal was similar to biofilm dispersal by gel containing DspB alone.

FIG. 53 is a bar graph showing biofilm dispersal activity of PF127 gel prepared in 50 mM phosphate buffer (buffer), 0.1% CPC alone, 20 μg/ml DspB alone and 20 μg/ml DspB plus 0.1% CPC. Results showed that DspB retained its activity for up to 2 h in the presence of CPC. Biofilm dispersal activity of gel containing DspB alone was similar to the gel containing DspB and CPC (P>0.05).

FIG. 54 is a bar graph showing activity of DspB in the presence of 0.1% CPC. DspB concentration used in mixture (DspB+CPC) was 100 μg/mL in PF127 gel prepared in 50 mM citrate buffer and it's % activity was calculated compared to the activity of 100 μg/mL DspB (considered 100% activity) as a control sample. Results showed that DspB was active for up to 2 h. *Indicate significant change (P≤0.05) in DspB activity compared to DspB control.

### Example 28—Benzalkonium Chloride (BZC) and DspB Gel

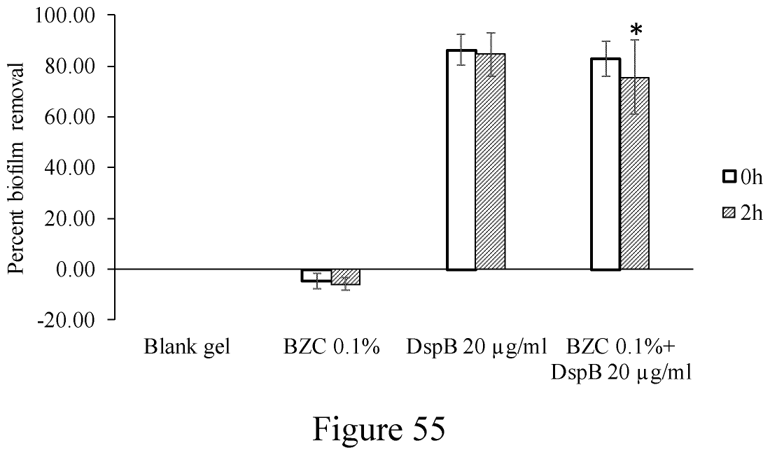

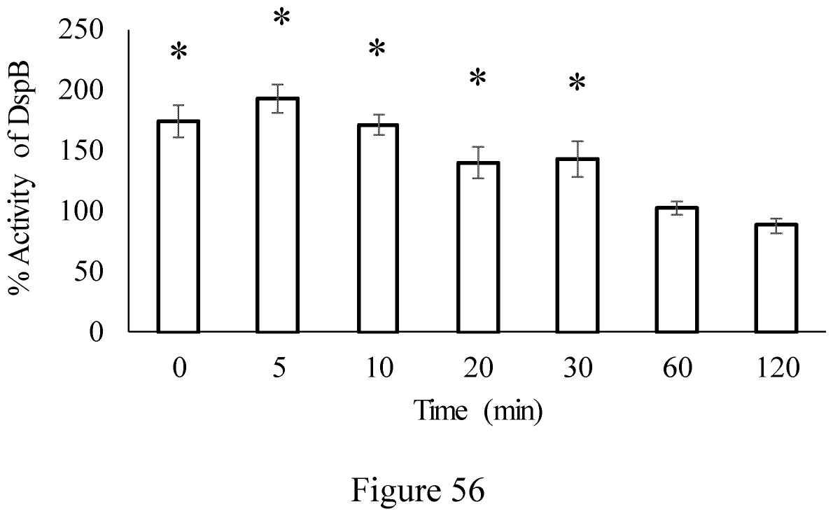

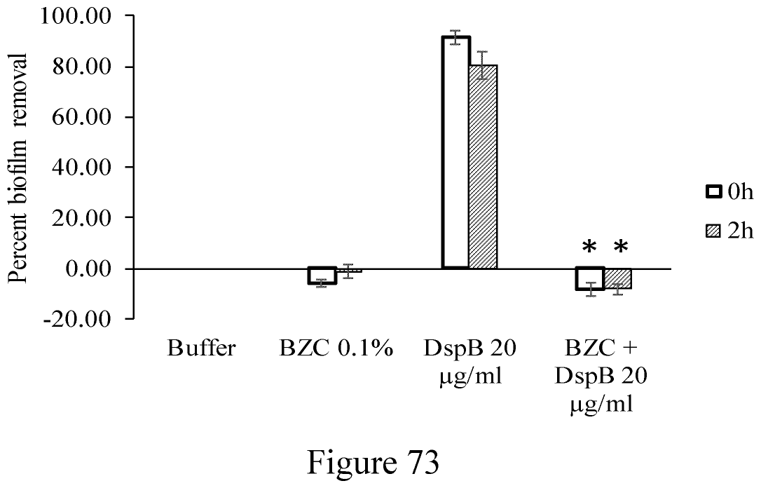

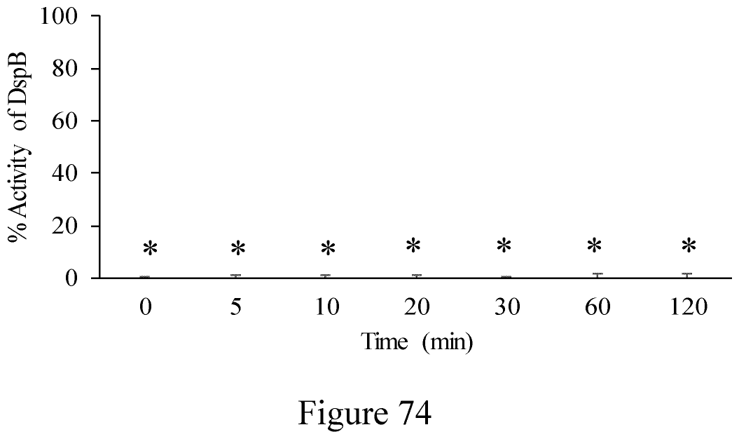

Based on biofilm dispersal assay (FIG. 55) and enzymatic assay (FIG. 56), DspB remained active for up to 2 h in the presence of BZC in wound gel. In contrast to results observed in buffer formulation (FIGS. 73 and 74), where DspB activity was lost by 0 h in the presence of BZC, DspB remained active for 2 h in gel formulation.

FIG. 55 is a bar graph showing biofilm dispersal activity of PF127 gel prepared in 50 mM phosphate buffer (buffer), 0.1% BZC alone, 20 μg/ml DspB alone and 20 μg/ml DspB plus 0.1% BZC. Results showed that DspB retained its activity for up to 2 h in the presence of BZC. Biofilm dispersal activity of gel containing DspB and BZC was similar to the gel containing DspB alone (P>0.05) at 0 h and significantly lower at 2 h (P≤0.05).

FIG. 56 is a bar graph showing activity of DspB in the presence of 0.1% BZC. DspB concentration used in mixture (DspB+BZC) was 100 μg/mL in PF127 gel prepared in 50 mM citrate buffer and it's % activity was calculated compared to the activity of 100 μg/mL DspB (considered 100% activity) as a control sample. Results showed that DspB was active for up to 2 h. *Indicate significant change (P≤0.05) in DspB activity compared to DspB control.

### Example 29—Providone Iodine (PI) and DspB Gel

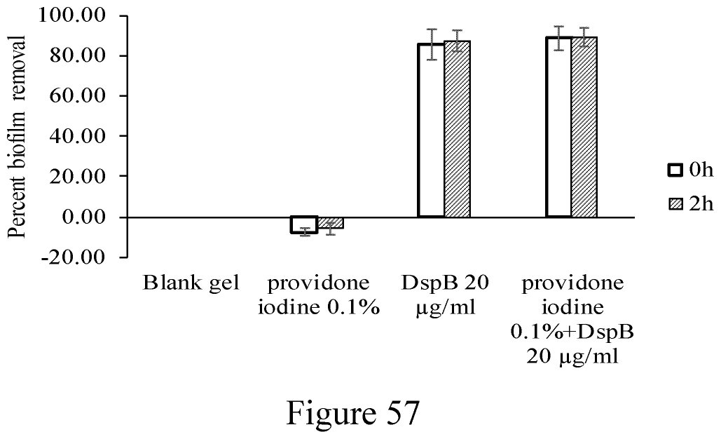

Based on biofilm dispersal assay (FIG. 57), DspB remained active for up to 2 h in the presence of PI in wound gel. In contrast to results observed in buffer formulation (FIG. 25), where DspB activity was lost by 2 h in the presence of PI, DspB remained active for 2 h in gel formulation, and biofilm dispersal was similar to biofilm dispersal by gel containing DspB alone.

FIG. 57 is a bar graph showing biofilm dispersal activity of PF127 gel prepared in 50 mM phosphate buffer (buffer), 0.1% active iodine (1% providone iodine) alone, 20 μg/ml DspB alone and 20 μg/ml DspB plus 0.1% iodine. Results showed that DspB retained its activity for up to 2 h in the presence of iodine. Biofilm dispersal activity of gel containing DspB and iodine was similar to the gel containing DspB alone (P>0.05).

### Example 30—Gentamicin Sulfate and DspB Gel

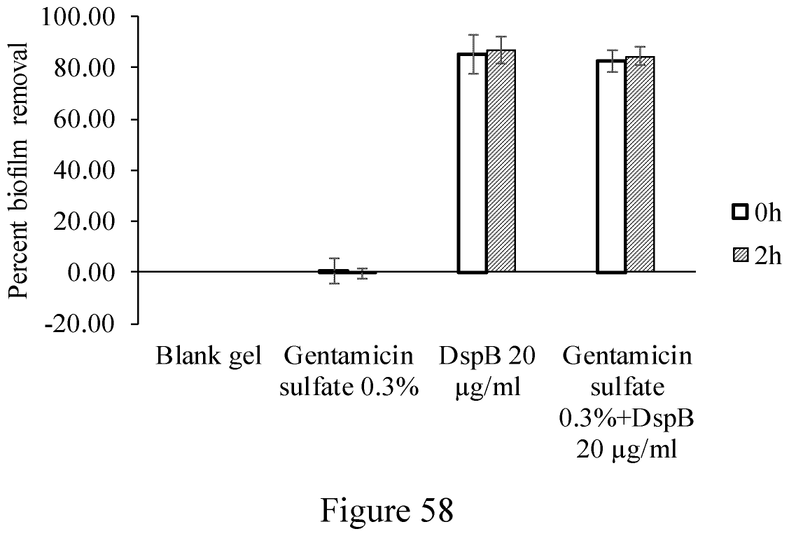

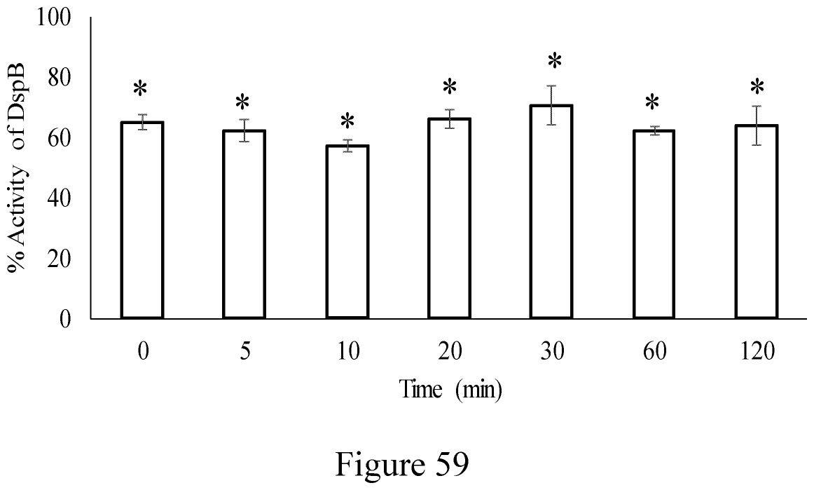

Based on biofilm dispersal assay (FIG. 58) and enzymatic assay (FIG. 59), DspB remained active for up to 2 h in the presence of gentamicin sulfate in wound gel.

FIG. 58 is a bar graph showing biofilm dispersal activity of PF127 gel prepared in 50 mM phosphate buffer (buffer), 0.3% gentamicin sulfate alone, 20 μg/ml DspB alone and 20 μg/ml DspB plus 0.3% gentamicin sulfate. Results showed that DspB retained its activity for up to 2 h in the presence of gentamicin sulfate. Biofilm dispersal activity of gel containing DspB and gentamicin sulfate was similar to the gel containing DspB alone (P>0.05).

FIG. 59 is a bar graph showing activity of DspB in the presence of 0.3% gentamicin sulfate. DspB concentration used in mixture (DspB+gentamicin sulfate) was 100 μg/mL in PF127 gel prepared in 50 mM citrate buffer and it's % activity was calculated compared to the activity of 100 μg/mL DspB (considered 100% activity) as a control sample. Results showed that DspB was active for up to 2 h. *Indicate significant change (P≤0.05) in DspB activity compared to DspB control.

### Example 31—Neomycin Sulphate and DspB Gel

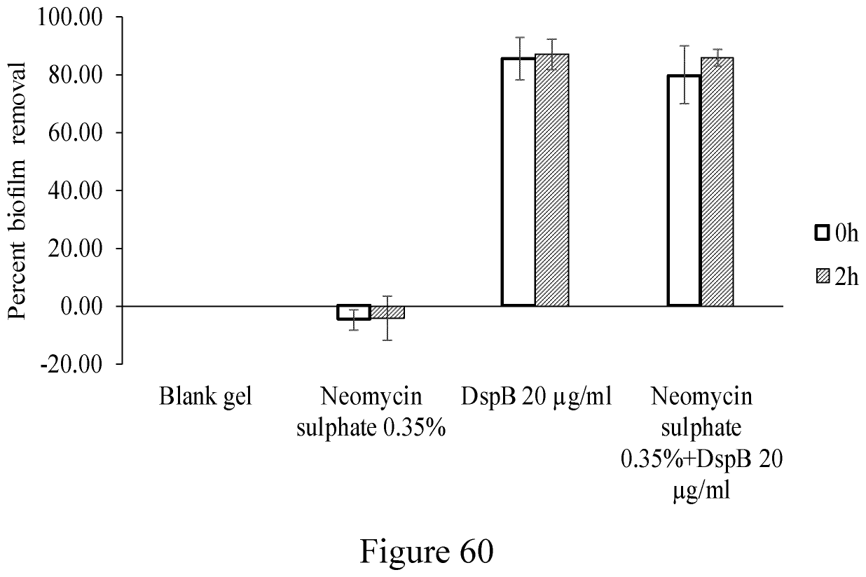

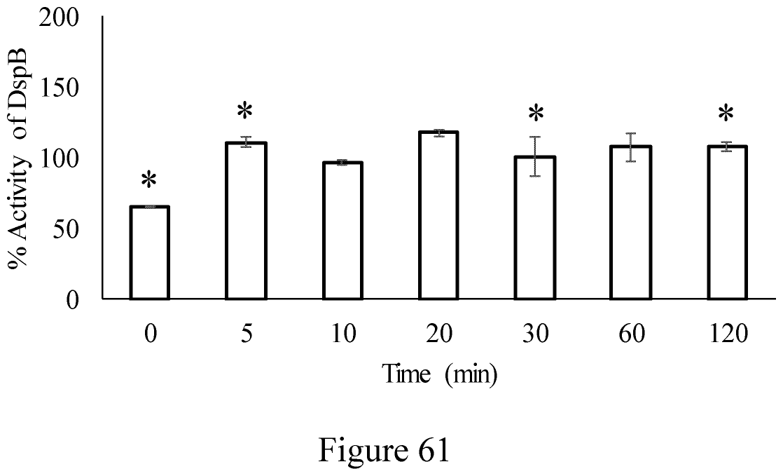

Based on biofilm dispersal assay (FIG. 60) and enzymatic assay (FIG. 61), DspB remained active for up to 2 h in the presence of neomycin sulphate in wound gel.

FIG. 60 is a bar graph showing biofilm dispersal activity of PF127 gel prepared in 50 mM phosphate buffer (buffer), 0.35% neomycin sulphate alone, 20 μg/ml DspB alone and 20 μg/ml DspB plus 0.35% neomycin sulphate. Results showed that DspB retained its activity for up to 2 h in the presence of neomycin sulphate. Biofilm dispersal activity of gel containing DspB and neomycin sulphate was similar to the gel containing DspB alone (P>0.05).

FIG. 61 is a bar graph showing activity of DspB in the presence of 0.35% neomycin sulphate. DspB concentration used in mixture (DspB+neomycin sulphate) was 100 μg/mL in PF127 gel prepared in 50 mM citrate buffer and it's % activity was calculated compared to the activity of 100 μg/mL DspB (considered 100% activity) as a control sample. Results showed that DspB was active for up to 2 h. *Indicate significant change (P≤0.05) in DspB activity compared to DspB control.

### Example 32—Polymyxin B Sulfate and DspB Gel

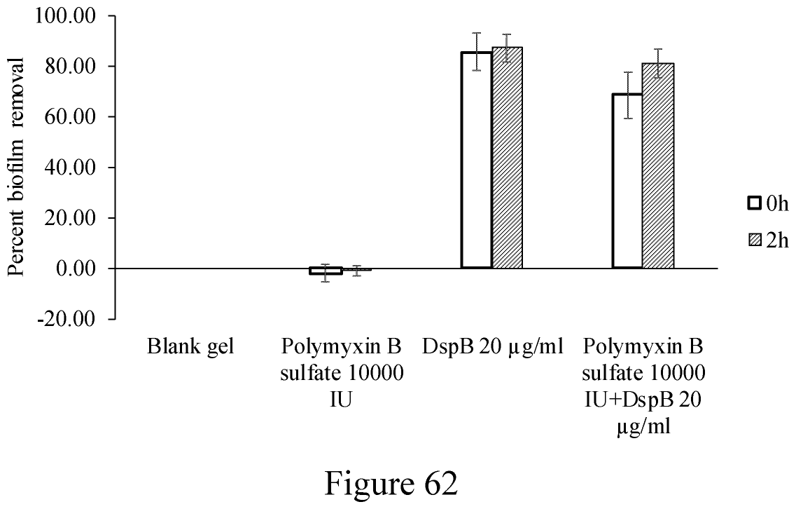

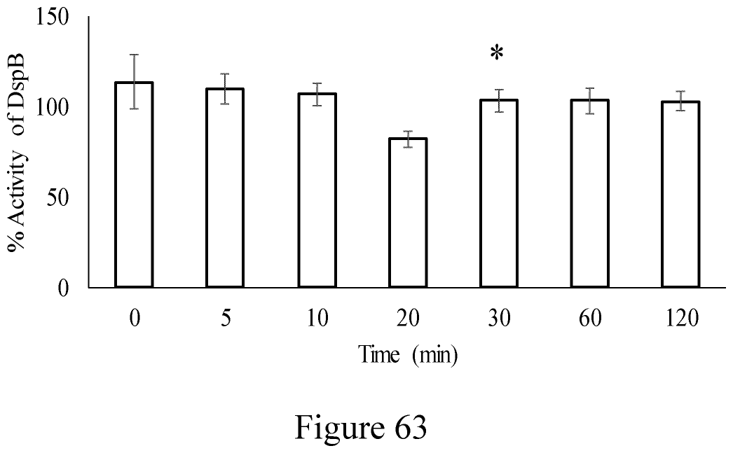

Based on biofilm dispersal assay (FIG. 62) and enzymatic assay (FIG. 63), DspB remained active for up to 2 h in the presence of polymyxin B sulfate in wound gel.

FIG. 62 is a bar graph showing biofilm dispersal activity of PF127 gel prepared in 50 mM phosphate buffer (buffer), 10000 IU polymyxin B sulfate alone, 20 μg/ml DspB alone and 20 μg/ml DspB plus polymyxin B sulfate. Results showed that DspB retained its activity for up to 2 h in the presence of polymyxin B sulfate. Biofilm dispersal activity of gel containing DspB and polymyxin B sulfate was similar to the gel containing DspB alone (P>0.05).

FIG. 63 is a bar graph showing activity of DspB in the presence of 5000 IU (0.083%) Polymyxin B sulfate. DspB concentration used in mixture (DspB+Polymyxin B sulfate) was 100 μg/mL in PF127 gel prepared using 50 mM phosphate buffer and it's percent activity was calculated compared to the activity of 100 μg/mL DspB (considered 100% activity) as a control sample. Results showed that DspB was active in the presence of Polymyxin B sulfate for upto 2 h. *Indicate significant increase (P≤0.05) in DspB activity compared to DspB control.

### Example 33—Mupirocin and DspB Gel

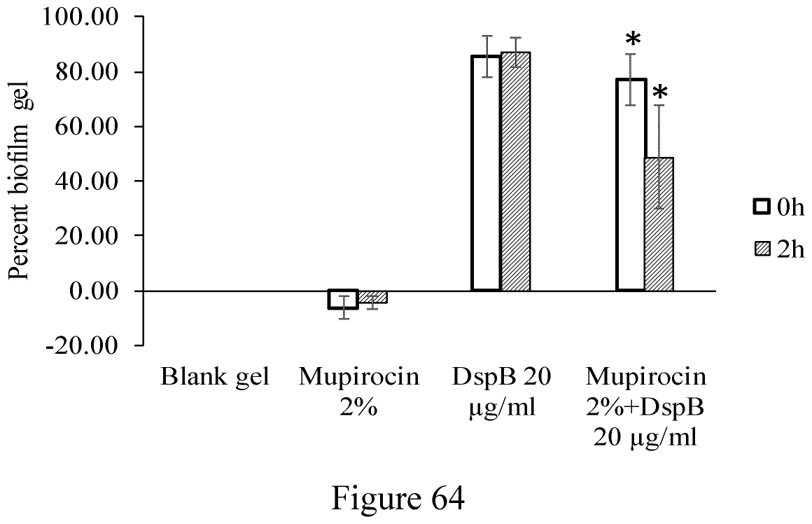

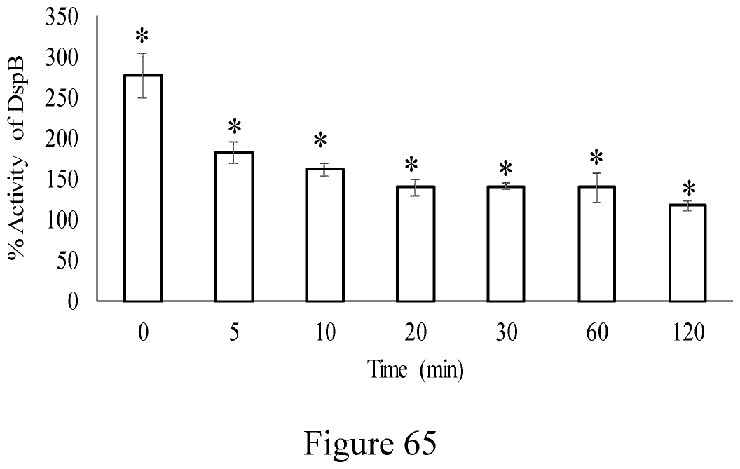

Based on biofilm dispersal assay (FIG. 64) and enzymatic assay (FIG. 65), DspB remained active for up to 2 h in the presence of Mupirocin in wound gel. Enzymatic assay showed that DspB activity remained >100% for up to 2 h.

FIG. 64 is a bar graph showing biofilm dispersal activity of PF127 gel prepared in 50 mM phosphate buffer (buffer), 2% mupirocin, 20 μg/ml DspB alone and 20 μg/ml DspB plus 2% mupirocin. Results showed that DspB retained its activity for up to 2 h in the presence of mupirocin. Biofilm dispersal activity of gel containing DspB and mupirocin was lower than to the gel containing DspB alone (P≤0.05).

FIG. 65 is a bar graph showing activity of DspB in the presence of 2% mupirocin. DspB concentration used in mixture (DspB+mupirocin) was 100 μg/mL in PF127 gel prepared in 50 mM citrate buffer and it's % activity was calculated compared to the activity of 100 μg/mL DspB (considered 100% activity) as a control sample. Results showed that DspB was active for up to 2 h. *Indicate significant change (P≤0.05) in DspB activity compared to DspB control.

### Example 34—Gramicidin and DspB Gel

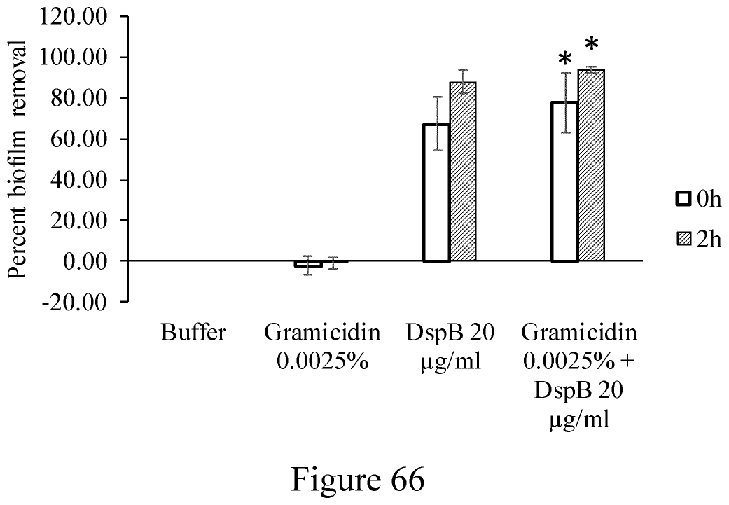

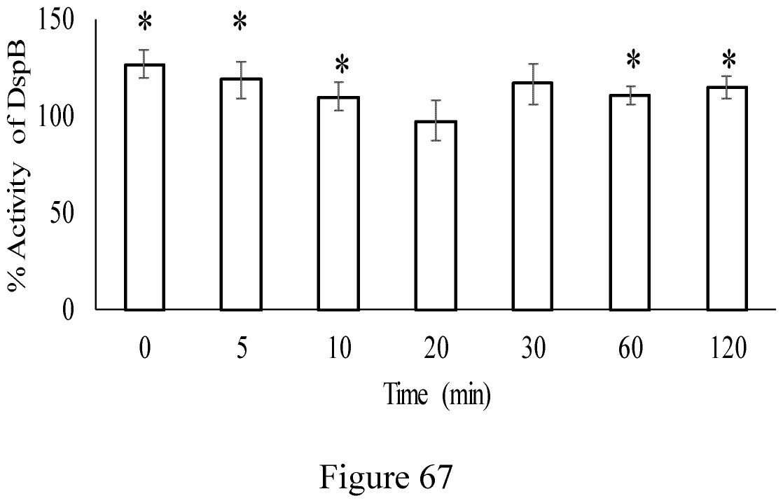

Based on biofilm dispersal assay (FIG. 66) and enzymatic assay (FIG. 67), DspB remained active for up to 2 h in the presence of gramicidin in wound gel. Enzymatic assay showed that DspB activity remained >100% for up to 2 h.

FIG. 66 is a bar graph showing biofilm dispersal activity of PF127 gel prepared in 50 mM phosphate buffer (buffer), 0.0025% gramicidin, 20 μg/ml DspB alone and 20 μg/ml DspB plus 0.0025% gramicidin. Results showed that DspB retained its activity for up to 2 h in the presence of mupirocin. Biofilm dispersal activity of gel containing DspB and mupirocin was higher than to the gel containing DspB alone (P≤0.05).

FIG. 67 is a bar graph showing activity of DspB in the presence of 0.0025% gramicidin. DspB concentration used in mixture (DspB+gramicidin) was 100 μg/mL in PF127 gel prepared in 50 mM citrate buffer and it's % activity was calculated compared to the activity of 100 μg/mL DspB (considered 100% activity) as a control sample. Results showed that DspB was active for up to 2 h. *Indicate significant change (P≤0.05) in DspB activity compared to DspB control.

### Example 35—Metronidazole and DspB Gel

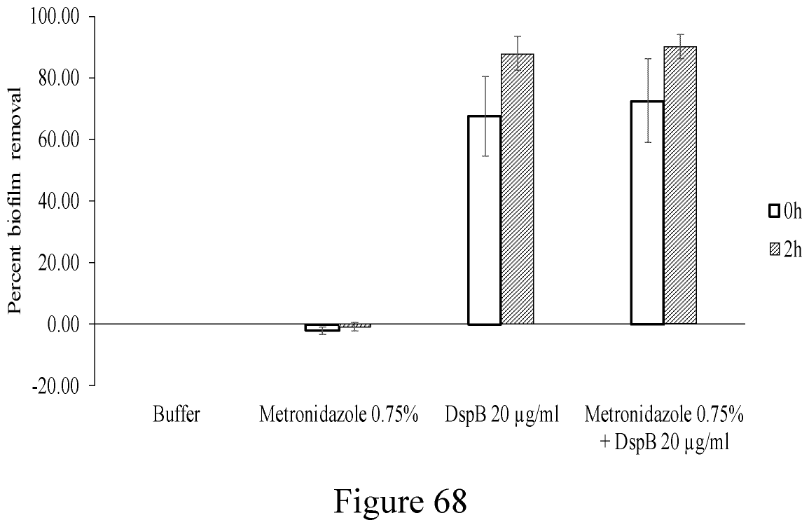

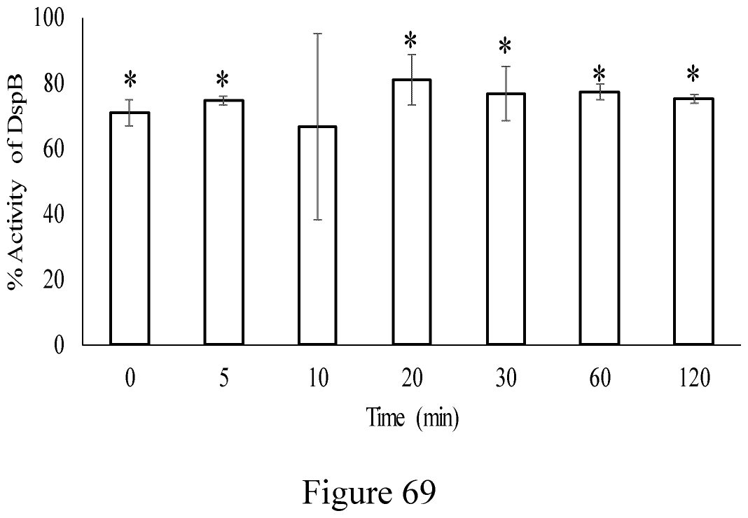

Based on biofilm dispersal assay (FIG. 68) and enzymatic assay (FIG. 69), DspB remained active for up to 2 h in the presence of metronidazole in wound gel.

FIG. 68 is a bar graph showing biofilm dispersal activity of PF127 gel prepared in 50 mM phosphate buffer (buffer), 0.75% metronidazole, 20 μg/ml DspB alone and 20 μg/ml DspB plus 0.75% metronidazole. Results showed that DspB retained its activity for up to 2 h in the presence of metronidazole. Biofilm dispersal activity of gel containing DspB and metronidazole was similar to the gel containing DspB alone (P>0.05).

FIG. 69 is a bar graph showing activity of DspB in the presence of 0.75% metronidazole. DspB concentration used in mixture (DspB+metronidazole) was 100 μg/mL in PF127 gel prepared in 50 mM citrate buffer and it's % activity was calculated compared to the activity of 100 μg/mL DspB (considered 100% activity) as a control sample. Results showed that DspB was active for up to 2 h. *Indicate significant change (P≤0.05) in DspB activity compared to DspB control.

The following antimicrobials are expected to give similar results: monoterpenes/plant extracts or essential oil components: Terpinen-4-ol.

The following microorganism are expected to give similar results: Staphylococcus aureus, Klebsiella sp., Citrobacter sp., Acinitobacter sp., and coagulase-negative staphylococci (CNS).

### Some Examples of Antimicrobials That Did Not Work Synergistically or Coactively With DspB

### Example 36—Berberine Hydrochloride

FIG. 70 is bar graph showing the no synergistic activity when biofilm-embedded S. epidermidis treated with no antimicrobials (50 mM citrate buffer), DspB at 20 μg/ml, Berberine hydrochloride (Berberine) at 0.025, 0.05, and 0.1%, DspB+Berberine at 0.025%, DspB+Berberine 0.05% and DspB and Berberine at 0.1% for 10 min at 37° C. *Indicate significant reduction (P≤0.05) in viable numbers compared to buffer or DspB control.

### Example 37—Cinnamic Acid

FIG. 71 is bar graph showing the increased susceptibility of biofilm-embedded S. epidermidis treated with no antimicrobials 50 mM citrate buffer (Buffer), DspB at 20 μg/ml, cinnamic acid at 0.05, 0.1, and 0.2%, DspB+cinnamic acid at 0.05%, DspB+cinnamic acid 0.1% and DspB and cinnamic acid at 0.2% for 10 min at 37° C. *Indicate significant reduction (P≤0.05) in viable numbers compared to buffer.

### Example 38—Cetylpyridinium Chloride

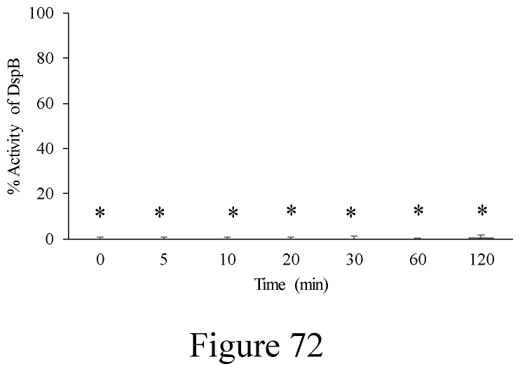

FIG. 72 is a bar graph showing activity of DspB in the presence of 0.1% Cetylpyridinium chloride (CPC). DspB concentration used in mixture (DspB+CPC) was 100 μg/mL in 50 mM phosphate buffer and it's % activity was calculated compared to the activity of 100 μg/mL DspB (considered 100% activity) as a control sample. Results showed that DspB was lost its activity in the presence of CPC. *Indicate significant reduction (P≤0.05) in DspB activity compared to DspB control.

### Example 39—Benzalkonium Chloride (BZC)

Data (FIGS. 73 and 74) showed that BZC inactivated DspB immediately after mixing (0 h)

FIG. 73 is a bar graph showing biofilm dispersal activity of, 50 mM phosphate buffer (buffer), 20 μg/ml DspB alone and 20 μg/ml DspB plus 0.1% BZC. Results showed that DspB lost its activity by 2 h in the presence of BZC. *Indicate significant reduction (P≤0.05) in biofilm dispersal compared to DspB control.

FIG. 74 is a bar graph showing activity of DspB in the presence of 0.1% BZC. DspB concentration used in mixture (DspB+BZC) was 100 μg/mL in 50 mM phosphate buffer and it's % activity was calculated compared to the activity of 100 μg/mL DspB (considered 100% activity) as a control sample. Results showed that DspB was lost its activity in the presence of BZC. *Indicate significant reduction (P≤0.05) in DspB activity compared to DspB control.

### Example 40—Refampin, Refapentine and Vancomycin

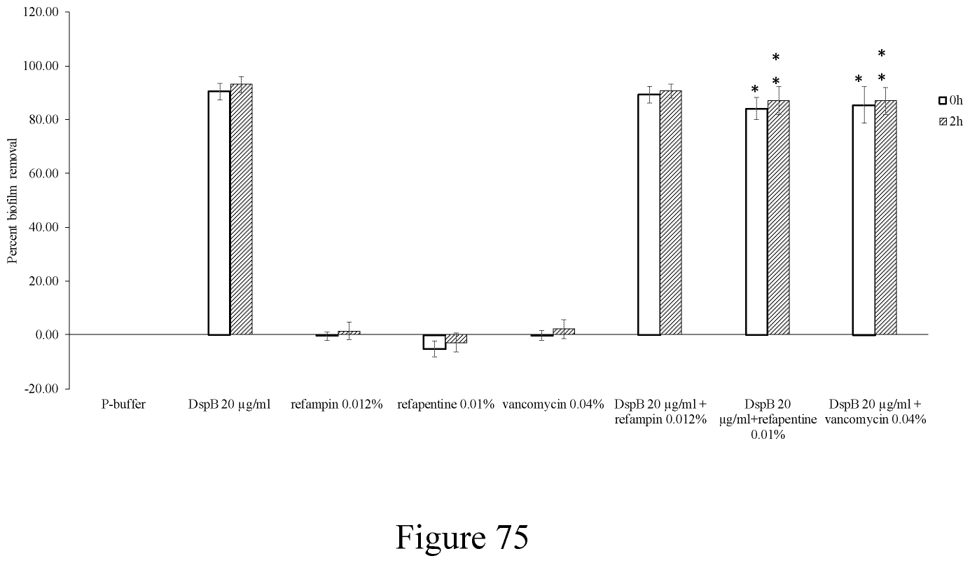

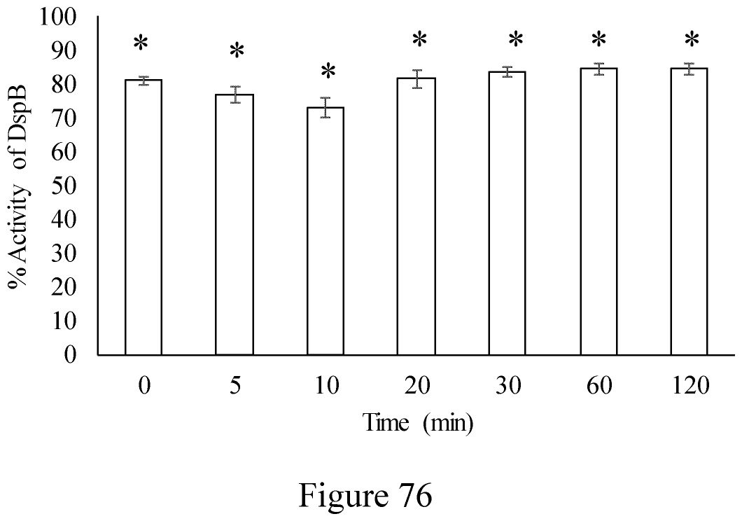

Based on biofilm dispersal assay (FIG. 75) and enzymatic assay (FIGS. 76-78), DspB remained active for up to 2 h when it was mixed together rifampin, rifapentine or vancomycin.

FIG. 75 is a bar graph showing biofilm dispersal activity of, 50 mM phosphate buffer (buffer), 20 μg/ml DspB alone, 0.012% rifampin alone, 0.01% rifapentine alone, 0.04% vancomycin alone, 20 μg/ml DspB plus 0.012% rifampin, 20 μg/ml DspB plus 0.012% rifapentine, and 20 μg/ml DspB plus 0.04% vancomycin. Results showed that DspB was active for up to 2 h in the presence of these antibiotics. *Indicate significant reduction (P≤0.05) in biofilm dispersal compared to DspB control.

FIG. 76 is a bar graph showing activity of DspB in the presence of 0.0024% rifampin. DspB concentration used in mixture (DspB+rifampin) was 100 μg/mL in 50 mM phosphate buffer and its percent activity was calculated compared to the activity of 100 μg/mL DspB (considered 100% activity) as a control sample. Results showed that DspB was active in the presence of rifampin for up to 2 h. *Indicate significant reduction (P≤0.05) in DspB activity compared to DspB control.

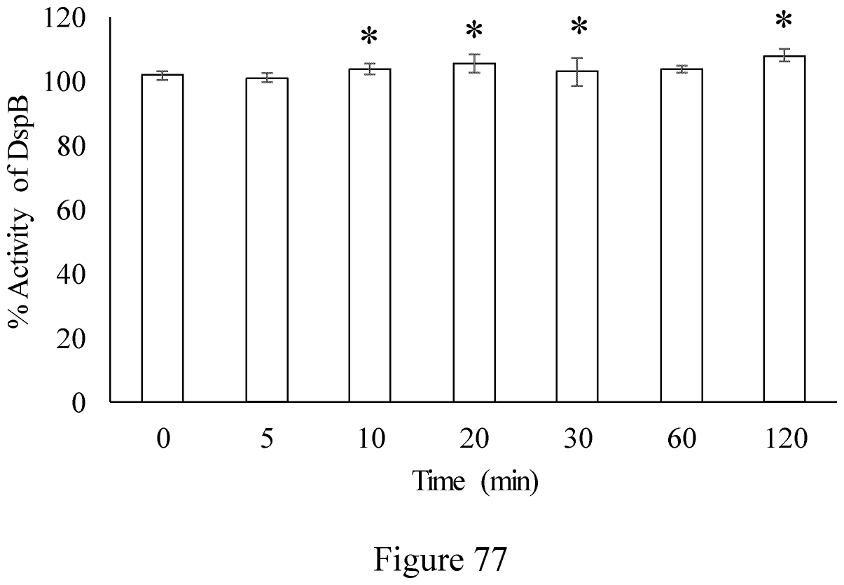

FIG. 77 is a bar graph showing activity of DspB in the presence of 0.006% rifapentine. DspB concentration used in mixture (DspB+rifapentine) was 100 μg/mL in 50 mM phosphate buffer and it's percent activity was calculated compared to the activity of 100 μg/mL DspB (considered 100% activity) as a control sample. Results showed that DspB was active in the presence of rifapentine for up to 2 h. *Indicate significant increase (P≤0.05) in DspB activity compared to DspB control.

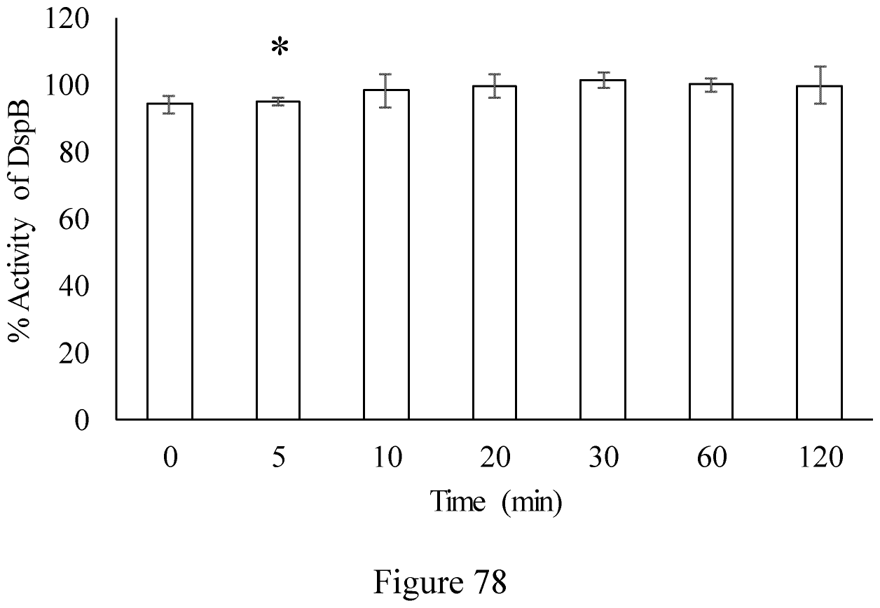

FIG. 78 is a bar graph showing activity of DspB in the presence of 0.04% vancomycin. DspB concentration used in mixture (DspB+vancomycin) was 100 μg/mL in 50 mM phosphate buffer and it's percent activity was calculated compared to the activity of 100 μg/mL DspB (considered 100% activity) as a control sample. Results showed that DspB was active in the presence of vancomycin for up to 2 h. *Indicate significant reduction (P≤0.05) in DspB activity compared to DspB control.

### Example 41—Manuka Honey

Based on enzymatic assay, manuka significantly honey (P≤0.05) enhanced activity of DspB for 4 h.

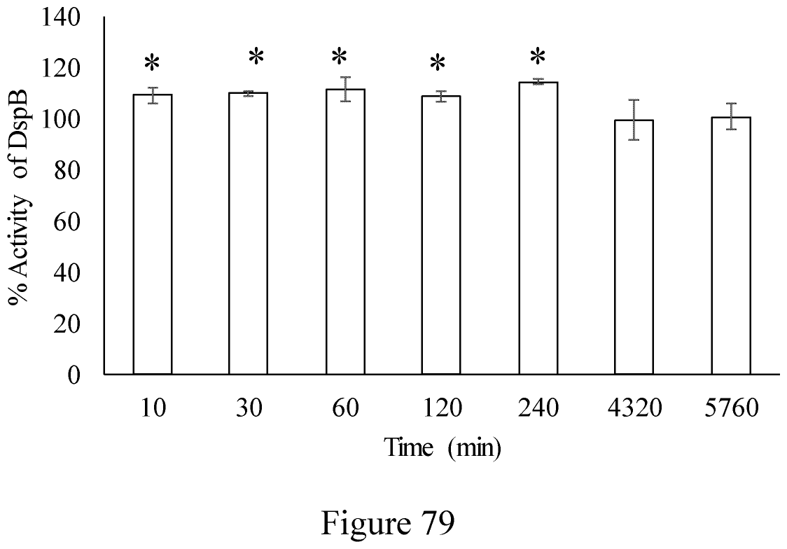

FIG. 79 is a bar graph showing activity of DspB in the presence of 20% manuka honey. DspB concentration used in mixture (DspB+manuka honey) was 100 μg/mL in 50 mM phosphate buffer and it's percent activity was calculated compared to the activity of 100 μg/mL DspB (considered 100% activity) as a control sample. Results showed that DspB was active in the presence of manuka honey for up to 96 h. *Indicate significant increase (P≤0.05) in DspB activity compared to DspB control.

Throughout the description, specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

TABLE 1
Summary of antimicrobials or antibiotics tested with DspB in buffer formulations
Summary of antimicrobials or antibiotics tested with DspB in buffer formulations

| | | Assay method: | | | | | |
|---|---|---|---|---|---|---|---|
| | | Microbio- | | Synergy or | Biofilm | | Reference for |
| | Concentrations | logical (M)/ | Concentration of | compatibility | inactiva- | Data | antimicrobials |
| Antimicrobial | tested | Enzymatic (E) | DspB tested | with DspB* | tion | presented | concentration selection |
| Polyhexamethylene | 0.00313-0.1% | M, E | M: 10-20 μg/ml | M: synergy | >5 log | FIGS. 1, 2, | Mikic et al. (2018); |
| biguanide (PHMB) | | | E: 100 μg/ml | E: compatible | CFU | 3 and 4 | Kamaruzzaman et al. |
| | | | | | | | (2017) |
| Polyaminopropyl | 0.05-0.2% | M | 20 μg/ml | M: synergy | >5 log | FIG. 5 | Rembe et al. (2016), |
| biguamide (PAPB) | | | | | CFU | | Ortega-Pe\grave{n}a et al., |
| | | | | | | | (2017) |
| Alexidine | 0.0125-0.05% | M | 20 μg/ml | M: synergy | >5 log | FIG. 6 | Machado Silveira et al. |
| dihydrochloride | | | | | CFU | | (2013) |
| (Alexidine) | 0.08-0.1% | M | M: 20 μg/ml | M: synergy | >5 log | FIGS. 7, 8, | Nostro et al. (2007) |
| Carvacrol | | | E: 100 μg/ml | E: synergy | CFU | 9 and 10 | |
| Thymol | 0.05-0.1% | M M, E | 20 μg/ml | M: synergy | >5 log | FIG. 11 | Nostro et al. (2007) |
| Cinnamaldehyde | 0.05-0.2% | | M: 20 μg/ml | M: synergy | CFU > | FIGS. 16, | Visvalingam et al. |
| | | | E: 100 μg/ml | E: synergy | 2 < 5 | 17, 18, and | (2017); Al-Bayati et al. |
| | | | | | log CFU | 19 | (2009) |
| Cinnamic acid | 0.05-0.2% | M, E | 20 μg/ml | M: no synergy | <0.5 log | FIG. 71 | Guzman (2014) |
| | | | | | CFU | | |
| Chicroxylenol | 0.05-0.2% | M, E | M: 20 μg/ml | M: synergy | >5 log | FIGS. 12, | Dankert and Schut. |
| | | | E: 100 μg/ml | E: synergy | CFU | 13, 14, and | (1976); Bloomfield and |
| | | | | | | 15 | Loonev. 1992. J. Appl. |
| | | | | | | | Microbiol. 73: 87-93. |
| Octenidine | 0.001-0.1% | M, E | M: 100-200 μg/ml | M: synergy | >5 log | FIGS. 20, | Koburger et al. (2010) |
| dihydrochloride | | | E: 100 μg/ml | E: no synergy | CFU | 21, 22 and | |
| | | | | | | 23 | |
| Nerol | 0.05-0.2% | M | 20 μg/ml | M: no synergy | <0.5 log | Not | Jirovetz et al. 2007. |
| | | | | | CFU | presented | Journal of Essential Oil |
| | | | | | | | Research 19: 288-291 |
| Nerolidol | 0.05-0.2% | M | 20 μg/ml | M: no synergy | <0.5 log | Not | Chan et al. 2016. |
| | | | | | CFU | presented | Molecules. 21(5): 529. |
| Berberine | 0.025-0.1% | M | 20 μg/ml | M: no synergy | <5 > 2 log | FIG. 70 | Wang et al. 2009. |
| hydrochloride | | | | | CFU | | International Journal of |
| | | | | | | | Antimicrobial Agents. |
| | | | | | | | 34: 60-66. |
| Geranyl acetone | 0.1-0.2% | M | M: 20 μg/ml | M: no synergy | <0.5 log | Not | Bonikowski et al. 2015. |
| | | | | | CFU | presented | Flavour Fragt J. 30: 238- |
| | | | | | | | 244 |
| Amitripryline | 0.05-0.2% | E | E: 20 μg/ml | E: no synergy | Not tested | Not | Mandal et al. 2010. Braz |
| hydrochloride | | | | | | presented | J Microbiol. 41: 635- |
| | | | | | | | 645. |
| Nisin | 0.001-0.004% | M | M: 20 μg/ml | M: no synergy | <0.5 log | Not | Severina et al. 1998. J |
| | | | | | CFU | presented | Antimicrob Chemother. |
| | | | | | | | 41: 341-347. |
| BisEDT | 0.007-0.03% | M | E: 20 μg/ml | M: no synergy | <1 log | Not | Domenico et al. 2001. |
| | | | | | CFU | presented | Antimicrob Agents |
| | | | | | | | Chemother. 45: 1417- |
| | | | | | | | 1421. |
| Sertraline | 0.00625-0.1% | E | E: 20 μg/ml | E: no synergy | Not tested | Not | Munoz-Bellido et al. |
| | | | | | | presented | 2000. International |
| | | | | | | | Journal of Antimicrobial |
| | | | | | | | Agents. 14: 177-180. |
| Oleanolic acid | 0.00125-0.02% | E | E: 20 μg/ml | E: no synergy | Not tested | Not | Kim et al 2012. Korean |
| | | | | | | presented | Journal of Microbiology. |
| | | | | | | | 48: 212-215. |
| Ursolic acid | 0.0125-0.1% | E | E: 20 μg/ml | E: no synergy | Not tested | Not | Fontanay et al. 2008. |
| | | | | | | presented | Journal of |
| | | | | | | | Ethnopharmacology |
| | | | | | | | 120: 272-276. |
| Cetylpyridinium | 0.1% | Microbiological | M: 20 μg/ml | M: compatible | Not tested | FIGS. 24 | FDA. 1994. Oral health |
| chloride | | and enzymatic | E: 100 μg/ml | E: no synergy | | and 72 | care drug products for |
| | | | | | | | over-the-counter human |
| | | | | | | | use. 59(27): 6084-6124. |
| Benzalkonium | 0.1% | Microbiological | M: 20 μg/ml | M: no synergy | Not tested | FIGS. 73 | FDA. 1994. Oral health |
| chloride | | and enzymatic | E: 100 μg/ml | E: no synergy | | and 74 | care drug products for |
| | | | | | | | over-the-counter human |
| | | | | | | | use. 59(27): 6084-6124. |
| | | | | | | | BlastX wound gel |
| Providone indine | 0.1% | Microbiological | M: 20 μg/ml | M: compatible | Not tested | FIG. 25 | Lipskey and Hoey. 2009. |
| | active | | | | | | Clinical Infectious |
| | iodme | | | | | | Diseases. 49: 1541-1549. |
| Manuka Honey | 20% | Enzymatic | E: 20% | E: synergy | Not tested | FIG. # | Johnston et al. 2018. |
| | | | | | | | AIMS Microbiol. 4: 655- |
| | | | | | | | 664. |
| Gentamicin | 0.3% | Microbiological | M: 20 μg/ml | M: compatible | Not tested | FIGS. 26 | Gentak Ointment |
| sulfate | | and enzymatic | E: 100 μg/ml | E: compatible | | and 27 | |
| Neomycin | 0.35% | Microbiological | M: 20 μg/ml | M: compatible | Not tested | FIGS. 28 | Antibiotic Ointment |
| sulphate | | and enzymatic | E: 100 μg/ml | E: compatible | | and 29 | |
| Polymyxin B | 5000- | Microbiological | M: 20 μg/ml | M: compatible | Not tested | FIGS. 30 | Antibiotic drop: |
| sulfate | 10000 | aud enzymatic | E: 100 μg/ml | E: synergy | | and 31 | Gramicidin D |
| | IU | | | | | | (0.025 mg/g) + |
| | | | | | | | Polymyxin B sulfate |
| | | | | | | | (10000 unit) |
| Mupirocin | 2% | Microbiological | M: 20 μg/ml | M: compatible | Not tested | FIGS. 32 | Bactroban Ointment |
| | | and enzymatic | E: 100 μg/ml | E: compatible | | and 33 | |
| Gramicidin | 0.0025% | Microbiological | M: 20 μg/ml | M: compatible | Not tested | FIGS. 34 | Antibiotic drop: |
| | | and enzymatic | E: 100 μg/ml | E: compatible | | and 35 | Gramicidin D |
| | | | | | | | (0.025 mg/g) + |
| | | | | | | | Polymyxin B sulfate |
| | | | | | | | (10000 unit) |
| Metronidazole | 0.75% | Microbiological | M: 20 μg/ml | M: compatible | Not tested | FIGS. 36 | Topical cream: 7.5 mg/ |
| Refampin | 0.0024%- | and enzymatic | E: 100 μg/ml | E: compatible | Not tested | and 37 | 1 g Albano et al. 2019. |
| | 0.012% | Microbiological | M: 0.012% | M: compatible | | FIG ## | Antimicro. Agents |
| | | and enzymatic | E: 0.0024% | E: compatible | | | Chemoth. 63: e00959-19. |
| | | | | | | | www.drugs.com |
| Refapentine | 0.006-0.01% | Microbiological | M: 0.01% | M: compatible | Not tested | FIG. ## | Albano et al. 2019. |
| | | and enzymatic | E: 0.006 | E: synergy | | | Antimicro. Agents |
| | | | | | | | Chemoth. 63: e00959-19. |
| | | | | | | | www.drugs.com |
| Vancomycin | 0.04% | Microbiological | M: 0.04% | M: compatible | Not tested | FIG. ## | Hair et al. 2018 bioRxiv |
| | | and enzymatic | E: 0.04 | E: compatible | | | 337436. www.drugs.com |

*For microbiological biofilm kill assay, synergy is concluded when numbers of bacteria inactivated by DspB and antimicrobial combination was significantly (P ≤ 0.05) higher than numbers of bacteria inactivated by total of DspB alone and antimicrobial alone. For enzymatic assay, synergy was concluded when percent enzymatic activity of DspB and antimicrobial/antibiotic was statistically higher than DspB alone. Compatibility is concluded when DspB retained its activity immediately after mixing or for up to 2 h in the presence of antimicrobial or antibiotic.

TABLE 2
Summary of antimicrobials or antibiotics tested with DspB in gel formulations
Summary of antimicrobials or antibiotics tested with DspB in gel formulations

| | | Assay method: | | Synergy or | | |
|---|---|---|---|---|---|---|
| | Concentrations | Microbiological (M)/ | Concentration | compatibility | Biofilm | Data |
| Antimicrobial | tested | Enzymatic (E) | of DspB tested | with DspB* | inactivation | presented |
| Polyhexamethylene | 0.1% | M, E | M: 20 μg/ml | M: synergy | >1->5log | FIGS. 39, 40, |
| biguanide (PHMB) | | | E: 100 μg/ml | E: compatible | | 41, 42, 43, 44 |
| Polyaminopropyl | 0.1% | M | M: 20 μg/ml | M: synergy | >1->5 log | FIG. 38 |
| biguanide (PAPB) | | | | | | |
| Carvacrol | 0.1% | M | M: 20 μg/ml | M: compatible | Not tested | FIGS. 45, 46 |
| | | | E: 100 μg/ml | E: synergy | | |
| Cinnamaldehyde | 0.2% | M, E | M: 20 μg/ml | M: compatible | Not tested | FIGS. 46, 47 |
| | | | E: 100 μg/ml | E: synergy | | |
| Chloroxylenol | 0.05-0.2% | M, E | M: 20 μg/ml | M: compatible | Not tested | FIGS. 48, 49 |
| | | | E: 100 μg/ml | E: synergy | | |
| Octenidine | 0.001-0.1% | M, E | M: 100-200 μg/ml | M: compatible | Not tested | FIGS. 50, 51 |
| dihydrochloride | | | E: 100 μg/ml | E: compatible | | |
| Cetylpyridinium | 0.1% | Microbiological and | M: 20 μg/ml | M: compatible | Not tested | FIGS. 52, 53 |
| chloride | | enzymatic | E: 100 μg/ml | E: compatible | | |
| Benzalkonium | 0.1% | Microbiological and | M: 20 μg/ml | M: compatible | Not tested | FIGS. 54, 55 |
| chloride | | enzymatic | E: 100 μg/ml | E: compatible | | |
| Providone | 0.1% | Microbiological | M: 20 μg/ml | M: compatible | Not tested | FIG. 56 |
| iodine | active | | | | | |
| | iodine | | | | | |
| Gentamicin | 0.3% | Microbiological and | M: 20 μg/ml | M: compatible | Not tested | FIGS. 57, 58 |
| sulfate | | enzymatic | E: 100 μg/ml | E: compatible | | |
| Neomycin | 0.35% | Microbiological and | M: 20 μg/ml | M: compatible | Not tested | FIGS. 59, 60 |
| sulphate | | enzymatic | E: 100 μg/ml | E: Yes | | |
| Polymyxin B | 5000- | Microbiological and | M: 20 μg/ml | M: compatible | Not tested | FIGS. 61, 62 |
| sulfate | 10000 IU | enzymatic | E: 100 μg/ml | E: compatible | | |
| Mupirocin | 2% | Microbiological and | M: 20 μg/ml | M: compatible | Not tested | FIGS. 63, 64 |
| | | enzymatic | E: 100 μg/ml | E: synergy | | |
| Gramicidin | 0.0025% | Microbiological and | M: 20 μg/ml | M: compatible | Not tested | FIGS. 65, 66 |
| | | enzymatic | E: 100 μg/ml | E: synergy | | |
| Metronidazole | 0.75% | Microbiological and | M: 20 μg/ml | M: compatible | Not tested | FIGS. 67, 68 |
| | | enzymatic | E: 100 μg/ml | E: compatible | | |

*For microbiological biofilm kill assay, synergy is concluded when numbers of bacteria inactivated by DspB and antimicrobial combination was significantly (P ≤ 0.05) higher than numbers of bacteria inactivated by total DspB alone and antimicrobial alone. For enzymatic assay, synergy was concluded when percent enzymatic activity was of DspB and antimicrobial/antibiotic combination was statistically higher than enzymatic activity of DspB alone. Compatibility is concluded when DspB retained its activity immediately after mixing or for up to 2 h in the presence of antimicrobial or antibiotic.

TABLE 4
Antimicrobial or antibiotics and their classes

| | | Other compounds |
|---|---|---|
| Tested compounds | Family/class | in this group |
| Polyhexamethylene | Biguanides | chlorhexidine |
| biguanide (PHMB) | | |
| Polyaminopropyl | | |
| biguanide (PAPB) | | |
| Alexidine | | |
| Carvacrol | Monoterpenes/plant | Limonene |
| Thymol | extracts/Essential oil | Terpinen-4-ol |
| | components | |
| Berberine/Berberine | Quaternary ammonium | |
| hydrochloride | | |
| benzalkonium chloride | | |
| cetylpyridinium chloride | | |
| Chloroxylenol | Halophenols | |
| Cinnamaldehyde | Phenylpropanoid/essential | |
| | oil component | |
| Octenidine | Cationic surfactant | |
| dihydrochloride | | |
| Mupirocin | Carboxylic acid | |
| Gentamycin Sulfate | Aminoglycosides | Amikacin |
| Neomycin Sulfate | | Kanamycin |
| | | Tobramycin |
| | | Streptomycin |
| Polymyxin B | Polypeptides | Bacitracin |
| Gramicidin | Ionophoric antibiotic | |
| Metronidazole | Nitroimidazoles | |
| Vancomycin | Glycopeptide | Teicoplanin |
| | | Ramoplanin |
| Rifampicin | RNA polymerase | |
| Rifabutin | inhibitor | |
| Rifapentine | | |

What is claimed is:

1. A method of preparing an antimicrobial in a composition with DispersinB to enhance biofilm dispersal and inactivation of biofilm embedded bacteria, the antimicrobial comprising a phenolic monoterpene compound selected from one or more of carvacrol and thymol.

2. A composition comprising DispersinB and an antimicrobial for enhancing biofilm dispersal and inactivation of biofilm embedded bacteria, the antimicrobial comprising a phenolic monoterpene compound selected from one or more of carvacrol and thymol.

3. The method of claim 1, wherein the antimicrobial is carvacrol and the concentration of carvacrol is up to 0.15%.

4. The method of claim 3, wherein the concentration of carvacrol is between 0.05 and 0.1%.

5. The method of claim 3, wherein the concentration of DspB is up to 100 pg/ml.

6. The method of claim 3, wherein the concentration of DspB is about 20 pg/ml.

7. The composition of claim 2, wherein the antimicrobial is carvacrol and the concentration of carvacrol is up to 0.15%.

8. The composition of claim 7, wherein the concentration of carvacrol is between 0.05 and 0.1%.

9. The composition of claim 7, wherein the concentration of DspB is up to 100 pg/ml.

10. The composition of claim 7, wherein the concentration of DspB is about 20 pg/ml.

11. The method of claim 1, wherein the antimicrobial is thymol and the concentration of thymol is up to 0.15%.

12. The method of claim 11, wherein the concentration of thymol is between 0.05 and 0.1%.

13. The method of claim 11, wherein the concentration of DspB is between 10-20 Mg/ml.

14. The method of claim 11, wherein the concentration of DspB is about 20 pg/ml.

15. The composition of claim 2, wherein the antimicrobial is thymol and the concentration of thymol is up to 0.15%.

16. The composition of claim 15, wherein the concentration of thymol is between 0.05 and 0.1%.

17. The composition of claim 15, wherein the concentration of DspB is between 10 and 100 pg/ml.

18. The composition of claim 15, wherein the concentration of DspB is about 20 pg/ml.
